(19) United States
(12) Patent Application Publication
(10) Pub. No.: US 2025/0051815 A1
(43) Pub. Date: Feb. 13, 2025

(54) COMPOSITIONS FOR ENZYMATIC POLYNUCLEOTIDE SYNTHESIS AND METHODS OF USE

(71) Applicant: Ansa Biotechnologies, Inc., Emeryville, CA (US)

(72) Inventors: Robert Nichols, Oakland, CA (US); Sebastian Palluk, Oakland, CA (US)

(21) Appl. No.: 18/719,075

(22) PCT Filed: Dec. 16, 2022
(86) PCT No.: PCT/US2022/081882

§ 371 Date: Jun. 12, 2024

Related U.S. Application Data
(60) Provisional application No. 63/290,310, filed on Dec. 16, 2021.

(51) Int. Cl. C12P 19/34

(52) U.S. Cl. CPC C12P 19/34

(57) ABSTRACT

Disclosed herein are compositions and methods for enzymatic nucleic acid synthesis. In particular, provided herein are nucleic acid synthesis reaction buffer compositions and associated methods for performing enzymatic nucleic acid synthesis comprising use of components and approaches that improve nucleotide synthesis.

### CROSS-REFERENCE TO RELATED APPLICATIONS

This application claims priority to and the benefit of U.S. Provisional Application No. 63/290,310, filed on Dec. 16, 2021, the entire disclosure of which is incorporated by reference herein for all purposes.

### REFERENCE TO A SEQUENCE LISTING

The instant application contains a Sequence Listing (XML file named ABB-008WO_SL.xml, generated on Dec. 13, 2022 and 3,150 bytes in size), which has been submitted electronically and is incorporated by reference herein.

### BACKGROUND

Enzymatic polynucleotide synthesis can be achieved through iterative rounds of template-independent nucleic acid polymerase (e.g. a terminal deoxynucleotidyl transferase) binding to a nucleic acid substrate (e.g. a DNA substrate), incorporation of a nucleotide (e.g. a protected or shielded nucleotide) to be added, followed by a deprotection step, allowing for future rounds of nucleotide incorporation. The speed, determined by enzymatic reaction kinetics, with which the DNA substrate is extended is a major limitation of the DNA synthesis field (FIG. 1A and FIG. 1B). With current methods, each cycle of nucleotide addition takes a prohibitive amount of time to ensure proper incorporation of the nucleotide in an extending polynucleotide. The time required for each nucleotide addition compounds with each extension reaction, and makes enzymatic nucleotide synthesis of desired polynucleotides limiting for many nucleic acid synthesis applications. Minimizing the time for each cycle is enabling for producing desired polynucleotides in a time and cost-effective manner.

There is a need in the art for improved enzymatic polynucleotide synthesis methods with conditions and buffer compositions that minimize the time of enzymatic polynucleotide extension reactions.

### SUMMARY

In one aspect, the disclosure provides methods of nucleic acid synthesis, comprising: providing a conjugate reagent comprising a plurality of polymerase-nucleotide conjugates, wherein said conjugates comprise a nucleotide or a modified nucleotide attached to a polymerase via a linker, and contacting a sample comprising a polynucleotide with said conjugate reagent, wherein said polymerase of the conjugate catalyzes an extension reaction comprising the covalent addition of a shielded nucleotide of the conjugate onto the 3′ hydroxyl of said polynucleotide in the presence of at least one divalent cation, and wherein the at least one divalent cation concentration is less than about 2500 μM. In some embodiments, the linker is a cleavable linker. In some embodiments, the methods described herein further comprise cleaving the linker. In some embodiments, the methods described herein further comprise repeating each of the steps as recited herein to synthesize a polynucleotide. In some embodiments, the polynucleotide comprises a pre-determined sequence.

In some aspects, the present disclosure provides methods of nucleic acid synthesis. In some embodiments, a method of nucleic acid synthesis, comprises providing a conjugate reagent comprising a plurality of polymerase-nucleotide conjugates, wherein said conjugates comprise a nucleotide attached to a polymerase via a linker; and providing a sample comprising a polynucleotide; contacting said sample with said conjugate reagent in a reaction volume, such that said sample and said conjugate reagent are combined and in the presence of one another, wherein said polymerase of the conjugate catalyzes an extension reaction comprising covalent addition of said nucleotide of said conjugate onto the 3′ hydroxyl of said polynucleotide, and wherein at least one divalent cation is present in said reaction volume; and wherein a total concentration of divalent cations present in the reaction volume is no greater than about 500 μM.

In some embodiments, the total concentration of divalent cations present in the reaction volume is no greater than about 250 μM, 125 μM, or about 50 μM.

In some embodiments, the extension reaction performed has a faster turnover rate than an extension reaction performed in the presence of the same or another divalent cation at a concentration of greater than about 1000 μM.

In some embodiments, the faster turnover rate is about is about 1 second faster, about 2 seconds faster, about 3 seconds faster, about 4 seconds faster, about 5 seconds faster, about 6 seconds faster, about 7 seconds faster, about 8 seconds faster, about 9 seconds faster, about 10 seconds faster, about 15 seconds faster, about 20 seconds faster, about 25 seconds faster, about 30 seconds faster, about 35 seconds faster, about 40 seconds faster, about 45 seconds faster, about 50 seconds faster, about 55 seconds faster, or about 60 seconds faster as compared to a reference reaction performed in the presence of the divalent cation at a concentration greater than about 1000 μM

In some embodiments, the divalent cation present at the highest concentration in the reaction volume, relative to the total concentration of divalent cations in the reaction volume, is cobalt (Co²⁺) or zinc (Zn²⁺).

In some embodiments, the at least one divalent cation is present at a concentration no greater than about 2500 μM, and the extension reaction performed in the presence of the concentration no greater than about 2500 μM has a faster turnover rate than an extension reaction performed in the presence of the same or another divalent cation at a concentration of greater than about 2500 μM. In some embodiments, the concentration no greater than about 2500 μM is about 1 μM, about 2.5 μM, about 5 μM, about 10 μM, about 15 μM, about 20 μM, about 25 μM, about 30 μM, about 35 μM, about 40 μM, about 45 μM, about 50 μM, about 100 μM, about 150 μM, about 200 μM, about 250 μM, about 300 μM, about 350 μM, about 400 μM, about 450 μM, about 500 μM, about 750 μM, about 1000 μM, about 1500 μM, about 2000 μM, or up to about 2500 μM.

In some embodiments, the faster turnover rate is between about 60 seconds faster and about 300 seconds faster as compared to a reference reaction not performed in the presence of a divalent cation concentration of no greater than about 2500 μM. In some embodiments, the faster turnover rate is about 1 second faster, about 2 seconds faster, about 3 seconds faster, about 4 seconds faster, about 5 seconds faster, about 6 seconds faster, about 7 seconds faster, about 8 seconds faster, about 9 seconds faster, about 10 seconds faster, about 15 seconds faster, about 20 seconds faster, about 25 seconds faster, about 30 seconds faster, about 35 seconds faster, about 40 seconds faster, about 45 seconds faster, about 50 seconds faster, about 55 seconds faster, or about 60 seconds faster as compared to a reference reaction not performed in the presence of a divalent cation concentration of no greater than about 2500 μM.

In some embodiments, the at least one divalent cation does not comprise Mg²⁺, Ca²⁺, Sr²⁺, Ba²⁺, Mn²⁺, Co²⁺, Fe²⁺, Ni²⁺, Cu²⁺, and Zn²⁺. In some embodiments, the at least one divalent cation is Co²⁺. In some embodiments, the at least one divalent cation is Zn²⁺. In some embodiments, the extension reaction is carried out in the absence of Mg²⁺.

In one aspect, the disclosure provides a method of synthesizing a polynucleotide comprising repeating one or more methods or one or more steps of a method provided herein one or more times.

In some embodiments, the polynucleotide comprises a pre-determined sequence.

In some embodiments, the polymerase is a template-independent polymerase. In some embodiments, the polymerase is selected from a Pol IV, a Pol μ, and a terminal deoxyribonucleotidyl transferase (TdT), or a variant thereof. In some embodiments, the polymerase is a TdT, or a variant thereof.

In some embodiments, the polymerase comprises a template-dependent polymerase. In some embodiments, the polymerase comprises a DNA polymerase. In some embodiments, the polymerase comprises a RNA polymerase.

In one aspect, the disclosure provides a nucleic acid synthesis reaction buffer comprising at least one divalent metal ion, wherein when used in a synthesis reaction, the concentration of the divalent metal ion present when the synthesis reaction is being performed is no greater than about than 2500 μM.

In some embodiments, the at least one divalent cation present in the synthesis reaction is less than about 1 μM, about 2.5 μM, about 5 μM, about 10 μM, about 15 μM, about 20 μM, about 25 μM, about 30 μM, about 35 μM, about 40 μM, about 45 μM, about 50 μM, about 100 μM, about 150 μM, about 200 μM, about 250 μM, about 300 μM, about 350 μM, about 400 μM, about 450 μM, about 500 μM, about 750 μM, about 1000 μM, about 1500 μM, about 2000 μM, or up to about 2500 μM.

In some embodiments, the at least one divalent cation is selected from Mg²⁺, Ca²⁺, Sr²⁺, Ba²⁺, Mn²⁺, Co²⁺, Fe²⁺, Ni²⁺, Cu²⁺, and Zn²⁺, or a combination thereof. In some embodiments, the at least one divalent cation is Mg²⁺. In some embodiments, the at least one divalent cation is Ca²⁺. In some embodiments, the at least one divalent cation is Sr²⁺. In some embodiments, the at least one divalent cation is Ba²⁺. In some embodiments, the at least one divalent cation is Mn²⁺. In some embodiments, the at least one divalent cation is Co²⁺. In some embodiments, the at least one divalent cation is Fe²⁺. In some embodiments, the at least one divalent cation is Ni²⁺. In some embodiments, the at least one divalent cation is Cu²⁺.

In some embodiments, the at least one divalent cation is Zn²⁺. In some embodiments, the nucleic acid synthesis reaction buffer further comprises tris(hydroxymethyl)aminomethane. In some embodiments, the nucleic acid synthesis reaction buffer further comprises 4-(2-hydroxyethyl)-1-piperazineethanesulfonic acid. In some embodiments, the nucleic acid synthesis reaction buffer further comprises a potassium salt. In some embodiments, the nucleic acid synthesis reaction buffer further comprises a nonionic surfactant. In some embodiments, the nucleic acid synthesis reaction buffer further comprises bovine serum albumin. In some embodiments, the nucleic acid synthesis reaction buffer further comprises sodium chloride. In some embodiments, the nucleic acid synthesis reaction buffer further comprises β-mercaptoethanol. In some embodiments, the nucleic acid synthesis reaction buffer further comprises glycerol.

In one aspect, the disclosure provides a method of nucleic acid synthesis, comprising: providing a conjugate reagent comprising a plurality of polymerase-nucleotide conjugates, wherein said conjugates comprise a nucleotide or a modified nucleotide attached (e.g., covalently linked) to a polymerase via a linker, and contacting a sample comprising a polynucleotide with said conjugate reagent, wherein said polymerase of the conjugate catalyzes an extension reaction comprising the covalent addition of a shielded nucleotide of the conjugate onto the 3′ hydroxyl of said polynucleotide in the nucleic acid synthesis reaction buffer of any one of the preceding claims.

In one aspect, the disclosure provides a method of nucleic acid synthesis, comprising providing a conjugate reagent comprising a plurality of polymerase-nucleotide conjugates, wherein said conjugates comprise a nucleotide covalently linked to a polymerase via a linker, and contacting a sample comprising a polynucleotide with said conjugate reagent, wherein said polymerase of the conjugate catalyzes an extension reaction comprising the covalent addition of the nucleotide of the conjugate onto the 3′ hydroxyl of said polynucleotide in the presence of a concentration of cobalt (Co²⁺), and, optionally, in the absence of Mg²⁺, wherein the concentration of Co²⁺ is selected from about 0.250 mM Co²⁺, about 0.125 mM Co²⁺, or about 0.050 mM Co²⁺, and wherein the extension reaction in the presence of the about 0.250 mM Co²⁺, about 0.125 mM Co²⁺, or about 0.050 mM Co²⁺ is faster than a reaction performed in the presence of a concentration of greater than 0.500 mM Co²⁺.

In one aspect, the disclosure provides a method of nucleic acid synthesis, comprising providing a conjugate reagent comprising a plurality of polymerase-nucleotide conjugates, wherein said conjugates comprise a nucleotide covalently linked to a polymerase via a linker, and contacting a sample comprising a polynucleotide with said conjugate reagent, wherein said polymerase of the conjugate catalyzes an extension reaction comprising the covalent addition of the nucleotide of the conjugate onto the 3′ hydroxyl of said polynucleotide in the presence of a concentration of zinc (Zn²⁺), and, optionally, in the absence of Mg²⁺, wherein the concentration of Zn²⁺ is selected from about 0.250 mM Zn²⁺, about 0.125 mM Zn²⁺, or about 0.050 mM Zn²⁺, and wherein the extension reaction in the presence of the about 0.250 mM Zn²⁺, about 0.125 mM Zn²⁺, or about 0.050 mM Zn²⁺ is faster than a reaction performed in the presence of a concentration of greater than 0.500 mM Zn²⁺.

In one aspect, the disclosure provides a method of reducing free nucleotide incorporation in a polynucleotide generated using a nucleic acid synthesis reaction comprising providing a conjugate reagent comprising a plurality of polymerase-nucleotide conjugates, wherein said conjugates comprise a nucleotide covalently linked to a polymerase via a linker, and contacting a sample comprising a polynucleotide with said conjugate reagent, wherein said polymerase of the conjugate catalyzes an extension reaction comprising the covalent addition of the nucleotide of the conjugate onto the 3′ hydroxyl of said polynucleotide in the presence of a concentration of cobalt (Co²⁺), and, optionally, in the absence of Mg²⁺, wherein the concentration of cobalt is about 0.050 mM Co²⁺, and wherein the incorporation of free nucleotides into a polynucleotide using a nucleotide extension reaction in the presence of the about 0.050 mM Co²⁺ is reduced than in a reaction performed in the presence of a concentration of greater than 0.125 mM Co²⁺.

In one aspect, the disclosure provides a method of improving a nucleotide synthesis reaction, the improvement comprising performing the nucleic acid synthesis in the presence of a divalent cation at a concentration of about 0.250 mM, about 0.125 mM, or about 0.050 mM of the divalent cation, wherein the speed of the extension reaction is greater than the speed of an extension reaction performed in the presence of a divalent cation at a concentration greater than about 0.500 mM.

### BRIEF DESCRIPTION OF THE DRAWINGS

The foregoing and other objects, features and advantages will be apparent from the following description of particular embodiments of the disclosure, as illustrated in the accompanying drawings in which like reference characters refer to the same parts throughout the different views. The drawings are not necessarily to scale, emphasis is instead placed upon illustrating the principles of various embodiments of the disclosure.

FIG. 1A and FIG. 1B are schemes illustrating an iterative cycle of enzymatic polynucleotide synthesis.

FIGS. 2A-2D are electropherograms showing results from analysis of products at different time points in an enzymatic polynucleotide extension reaction performed using a polymerase-nucleotide conjugate in a reaction buffer containing low cobalt acetate concentration (0.05 mM CoOAc; FIGS. 2B and 2D) or a standard cobalt acetate concentration (0.5 mM CoOAc; FIGS. 2A and 2C). FIG. 2E is a plot showing the quantification and analysis of products in FIGS. 2A-2D, and the associated calculated rates of reaction (k_{obs}).

FIGS. 3A-3L are electropherograms showing results from analysis of products at different time points in an enzymatic polynucleotide extension reaction performed using a polymerase-nucleotide conjugate in a reaction buffer containing a range of cobalt acetate concentrations (0.05 mM CoOAc, 0.125 mM CoOAc, 0.25 mM CoOAc, 0.75 mM CoOAc, 1.25 mM CoOAc, and 2.5 mM CoOAc). FIG. 3M is a plot showing the quantification and analysis of products in FIGS. 3A-3L, and the associated calculated rates of reaction (k_{obs}).

FIGS. 4A-4L are electropherograms showing results from analysis of products at different time points in an enzymatic polynucleotide extension reaction performed using a polymerase-nucleotide conjugate in a reaction buffer containing a range of zinc acetate (ZnOAc) concentrations (0.05 mM ZnOAc, 0.125 mM ZnOAc, 0.25 mM ZnOAc, 0.75 mM ZnOAc, 1.25 mM ZnOAc, and 2.5 mM ZnOAc). FIG. 4M is a plot showing the quantification and analysis of products in FIGS. 4A-4L, and the associated calculated rates of reaction (k_{obs}).

FIGS. 5A-5L are electropherograms showing results from analysis of products at different time points in an enzymatic polynucleotide extension reaction using a free polymerase and free nucleotide performed in a reaction buffer containing a range of cobalt acetate (CoOAc) concentrations (0.05 mM CoOAc, 0.125 mM CoOAc, 0.25 mM CoOAc, 0.75 mM CoOAc, 1.25 mM CoOAc, and 2.5 mM CoOAc). FIG. 5M is a plot showing the quantification and analysis of products in FIG. 5.

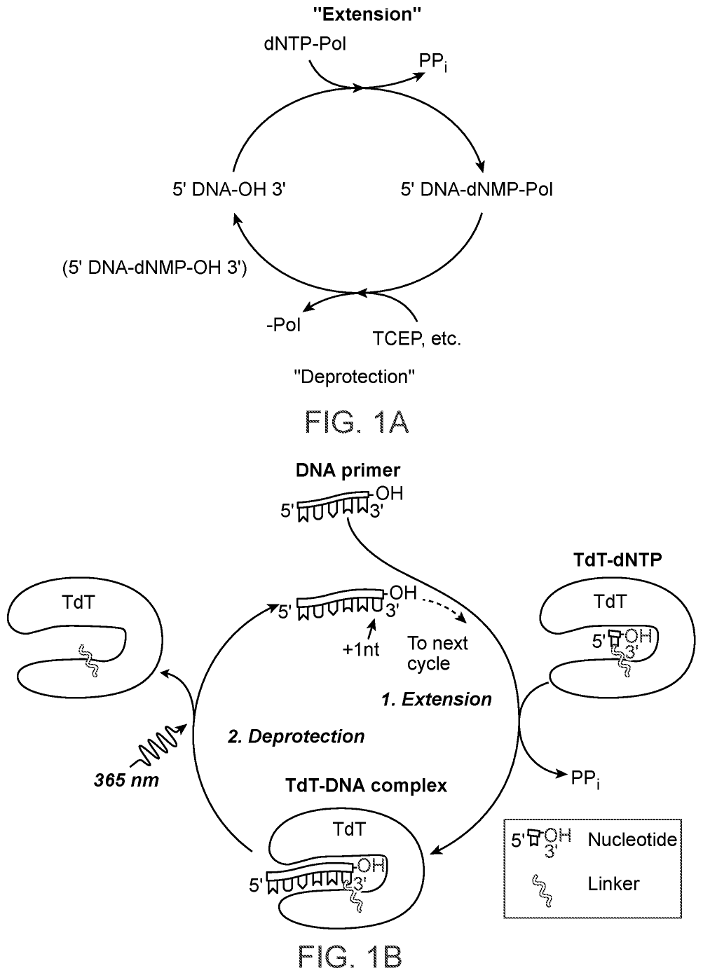

### DETAILED DESCRIPTION

The present disclosure provides insights based on a surprising discovery that rate of extension reactions using polymerase-nucleotide conjugates in polynucleotide synthesis can be increased by reducing the concentration of divalent cation concentrations present in the reactions as compared to standard divalent cation concentrations used in polynucleotide synthesis reactions using a free polymerase and free polynucleotide.

The disclosure provides compositions and methods related to the discovery. The details of various embodiments of the compositions and methods are set forth in the disclosure. Other features, objects, and advantages of the compositions and methods disclosed herein will be apparent from the description and the drawings, and from the claims.

As used herein, the term “nucleotide” refers to a molecule comprising a nucleoside and one or more phosphate groups. A “nucleoside” refers to a molecule comprising a nucleobase (e.g. adenine, thymine, cytosine, guanine, or uracil) and a five carbon sugar (e.g. ribose or 2′-deoxyribose). Exemplary nucleotides can be or comprise, without limitation, a nucleoside monophosphate, a nucleoside diphosphate, a nucleoside triphosphate, a nucleoside tetraphosphate, a nucleoside pentaphosphate, or a nucleoside hexaphosphate. As provided herein, TdT and TdT variants, can, in some embodiments, incorporate any nucleoside polyphosphate, including nucleotide analogs comprising modifications to the nucleobase.

As used herein, the term “nucleoside polyphosphate” is a “nucleotide” and may be called a “nucleotide polyphosphate.” For example, a “nucleotide triphosphate” and a “nucleoside triphosphate” both refer to a nucleotide comprising a nucleobase, a sugar, and a polyphosphate consisting of three linked phosphate groups.

As used herein, “non-termination” or “insertion” occurs when more than one nucleotide is added during a single step of a cyclic nucleotide extension. This can occur when an unshielded nucleotide with an uncleaved 5′ phosphate is added to an oligonucleotide.

As used herein, the term “protected nucleotide” or “shielded nucleotide” refers to a nucleotide that is sterically hindered by a tethered polymerase (or other entity or component such as, e.g., a blocking group) from a phosphatase capable of removing its 5′ phosphate. In some embodiments, such nucleotides are likely to inhibit subsequent nucleotide additions after having been added to an oligonucleotide and before removal of said tethered polymerase.

As used herein, the term “unprotected nucleotide” or “unshielded nucleotide” refers to a nucleotide that is not sterically hindered by a tethered polymerase (or other entity or component such as, e.g., a blocking group) from a phosphatase capable of removing its 5′ phosphate. some embodiments, an unshielded nucleotide may be tethered to a polymerase, such as in a misfolded polymerase or tethered at an incorrect position. An unshielded nucleotide may be untethered (or free) from a polymerase. Unshielded nucleotides that have not been exposed to phosphatase are more likely to be erroneously added to a polynucleotide as an insertion after a shielded nucleotide has been properly added.

### Nucleic Acid Synthesis

Disclosed herein are methods of nucleic acid synthesis. Nucleic acid synthesis can refer to synthesis, or generation of a product that is a nucleic acid molecule (i.e. a polynucleotide). The methods of nucleic acid synthesis can comprise stepwise synthesis, wherein nucleotides are inserted stepwise into a nucleic acid polymer or polynucleotide. A typical process for stepwise synthesis of a polynucleotide comprises adding nucleotides stepwise to a starter molecule (e.g., an initial oligonucleotide) via the cycled steps of: addition of a polymerase and a nucleotide (e.g. a polymerase-nucleotide conjugate) to an oligonucleotide and covalently incorporating the nucleotide to the 3′ end of the oligonucleotide catalyzed by the polymerase. Successful incorporation of a nucleotide to an oligonucleotide can be referred to as an “extension” or “extension reaction”.

In some embodiments, the polymerase and nucleotide are linked together (i.e. tethered) to form a conjugate (i.e. a polymerase-nucleotide conjugate). In a stepwise synthesis using the conjugate, the tethered nucleotide is covalently incorporated (i.e. is added) into the 3′ end of the oligonucleotide, which is catalyzed by the tethered polymerase. The tethered polymerase can stay tethered to the nucleotide following covalent incorporation. Covalent incorporation of a nucleotide can be referred to as an extension or an addition. The tethered polymerase can be cleaved from the inserted nucleotide to expose the 3′ end of the oligonucleotide. These steps can be repeated to synthesize a desired polynucleotide. The desired polynucleotide can have a pre-determined (i.e. pre-defined or target) sequence.

As will be understood to those of skill in the art, methods provided herein, such as nucleic acid synthesis, are performed in a reaction volume. Given context, the contents of the reaction volume may change before, after, and during the synthesis. For example, in some embodiments, the reaction volume comprises one or more of a buffer, polynucleotide, polymerase-nucleotide conjugate, nucleotide starter molecule, synthesis products, phosphatases, etc. In some such embodiments, a reaction volume may be prepared comprising only select components (e.g., a buffer and nucleotide starter molecule), and one or more additional components may be added to the volume at one or more subsequent times. In some embodiments, all components for a given synthesis may be added substantially simultaneously. In some embodiments, one or more components of a synthesis reaction may be pre-treated (e.g., pretreatment of a polymerase-nucleotide conjugate with a phosphatase) prior to being included in a reaction volume for a nucleic acid synthesis reaction.

Among other things, methods of nucleic acid synthesis disclosed herein are carried out in a reaction buffer composition. The reaction buffer composition is an aqueous solution. The reaction buffer composition comprises a set of components suitable for the stability of the polymerase, nucleotide, polymerase-nucleotide conjugates, starter molecule, nucleic acid molecule products, and any surface or matrix on which the methods disclosed herein are carried out. In some embodiments, methods of the present disclosure are performed on a solid surface, in a container such as a test tube, microplate, or other container having one or more vessels suitable for containing liquid, in a single dot on a solid surface and/or other matrix, etc. Further, the reaction buffer composition comprises a set of components suitable for carrying out catalytic steps (e.g. polynucleotide polymerization performed by a polymerase) described in the methods of nucleic acid synthesis described herein.

The conditions under which nucleic acid synthesis is carried out can be varied. For example, the times for carrying out each step in a stepwise nucleotide addition cycle can be varied to improve the purity of a plurality of products generated by the methods of nucleic acid synthesis described herein.

In some embodiments, a synthesis reaction occurs in the presence of one or more divalent cations. In some embodiments, the one or more divalent cations is selected from magnesium (Mg²⁺), calcium (Ca²⁺), strontium (Sr²⁺), barium (Ba²⁺), manganese (Mn²⁺), cobalt (Co²⁺), iron (Fe²⁺), nickel (Ni²⁺), copper (Cu²⁺), and/or zinc (Zn²⁺). In some embodiments, the one or more divalent cations is present at a concentration of 2.5 mM or less.

In some embodiments, the concentration of any given divalent cation present in a nucleic acid synthesis reaction volume is less than about 1 μM, about 2.5 μM, about 5 μM, about 10 μM, about 15 μM, about 20 μM, about 25 μM, about 30 μM, about 35 μM, about 40 μM, about 45 μM, about 50 μM, about 100 μM, about 150 μM, about 200 μM, about 250 μM, about 300 μM, about 350 μM, about 400 μM, about 450 μM, about 500 μM, about 1000 μM, about 1500 μM, about 2000 μM, or about 2500 μM. In some embodiments, the concentration of a divalent cation in a synthesis reaction is 0 μM or substantially 0 μM. In some such embodiments, such a divalent cation is considered absence from such a reaction.

In some embodiments, a concentration of divalent cations present in a synthesis reaction and/or a reaction volume includes a total concentration of divalent cations, which may be comprised of one or more divalent cations. In some embodiments, one or more divalent cations are selected from magnesium (Mg²⁺), calcium (Ca²⁺), strontium (Sr²⁺), barium (Ba²⁺), manganese (Mn²⁺), cobalt (Co²⁺), iron (Fe²⁺), nickel (Ni²⁺), copper (Cu²⁺), and zinc (Zn²⁺). In some embodiments, a concentration of divalent cations (e.g., in a reaction volume, e.g., of a synthesis reaction) includes one or more than one divalent cations. In some embodiments, one divalent cation (e.g., Co²⁺, e.g., Zn²⁺) is present at a concentration higher (e.g., by a certain percent) than any other divalent cations. In some embodiments, one divalent cation (e.g., Co²⁺, e.g., Zn²⁺) is present at the highest concentration relative to the concentration of one or more other divalent cations (e.g., Mg2+). In some embodiments, one divalent cation (e.g., Co²⁺, e.g., Zn²⁺) is present at the highest concentration by way of it being the only divalent cation present (i.e., absence of one or more additional divalent cations, such as, e.g., Mg²⁺, etc.). For instance, by way of non-limiting example, in some embodiments, a reaction volume may comprise or a synthesis reaction may occur in a total volume wherein the total concentration of divalent cations in the volume comprises at least two cations such as, e.g., Co²⁺ and Mg²⁺. In some such embodiments, one of those two divalent cations is present at a concentration that is higher (e.g., by about 1, 2, 3, 4, 5, 6, 7, 8, 9, 10 or more-fold) than the other divalent cation. In some embodiments, a reaction volume includes a total concentration of divalent cations comprised of a single divalent cation (e.g., Co²⁺, Zn²⁺).

Methods of nucleic acid synthesis disclosed herein can be used to generate a nucleic acid molecule product (i.e. a polynucleotide product). In some embodiments, the nucleic molecule product (i.e. polynucleotide product) has a target (i.e. pre-determined) sequence. A “target” or “pre-determined” sequence refers to a desired polynucleotide sequence that is intentionally produced by the method of nucleic acid synthesis. The pre-determined sequence can include any number of nucleotides comprising a nucleobase (e.g. adenine, thymine, guanine, cytosine, and/or uracil). In some embodiments, the nucleotide is a modified nucleotide (i.e. a nucleotide analog). In some embodiments, the nucleobase is a modified nucleobase. In some embodiments, the pre-determined sequence contains one or more designated positions which may be a random nucleobase. Inclusion of a position with a random nucleobase can be useful, for example, in introducing a randomized mutation into a polynucleotide product.

A nucleic acid molecule product or polynucleotide product generated by the methods described herein can contain a plurality of products. In some embodiments, the plurality of products comprises a nucleic acid molecule comprising the target (i.e. pre-determined) sequence. In some embodiments, the plurality of products comprises a nucleic acid molecule comprising a sequence that is not the target sequence. In some embodiments, the plurality of products comprises a nucleic acid molecule product comprising the target sequence and a nucleic acid molecule product that is not the target sequence. The “purity” of the plurality of products can refer to the ratio of the abundance of nucleic acid molecule products with the target sequence to the abundance of nucleic acid molecule products that do not have the target sequence, for example, taking into account all products and considering which proportion of products does or does not comprise a target sequence. Such purity measurements may be expressed in ways that represent the ratio of one or the other relative to all products, or one relative to the other such as with or without target sequences.

### Nucleic Acid Synthesis Reaction Buffer Compositions

The disclosure provides reaction buffer compositions for carrying out a polynucleotide extension reaction (i.e. nucleic acid synthesis reaction buffer). Polymerase enzymes for use in the disclosed methods require a divalent cation cofactor for carrying out the covalent addition of a nucleotide during enzymatic nucleic acid synthesis.

In some embodiments, the nucleic acid synthesis reaction buffer comprises at least one divalent metal ion. In some embodiments, the concentration of the at least one divalent metal ion is less than about 2500 μM. In some embodiments, the concentration of the at least one divalent cation is less than about 1 μM, about 2.5 μM, about 5 μM, about 10 μM, about 15 μM, about 20 μM, about 25 μM, about 30 μM, about 35 μM, about 40 μM, about 45 μM, about 50 μM, about 100 μM, about 150 μM, about 200 μM, about 250 μM, about 300 μM, about 350 μM, about 400 μM, about 450 μM, about 500 μM, about 1000 μM, about 1500 μM, about 2000 μM, or about 2500 μM. In some embodiments, the concentration of a given divalent cation in a nucleic acid synthesis reaction is 0 μM or substantially 0 μM. In some such embodiments, such a divalent cation is considered absent from such a reaction.

In some embodiments, the at least one divalent cation is selected from magnesium (Mg²⁺), calcium (Ca²⁺), strontium (Sr²⁺), barium (Ba²⁺), manganese (Mn²⁺), cobalt (Co²⁺), iron (Fe²⁺), nickel (Ni²⁺), copper (Cu²⁺), and zinc (Zn²⁺), or a combination thereof. In some embodiments, the at least one divalent cation is Mg²⁺. In some embodiments, the at least one divalent cation is Ca²⁺. In some embodiments, the at least one divalent cation is Sr²⁺. In some embodiments, the at least one divalent cation is Ba²⁺. In some embodiments, the at least one divalent cation is Mn²⁺. In some embodiments, the at least one divalent cation is Co²⁺. In some embodiments, the at least one divalent cation is Fe²⁺. In some embodiments, the at least one divalent cation is Ni²⁺. In some embodiments, the at least one divalent cation is Cu²⁺. In some embodiments, the at least one divalent cation is Zn²⁺.

In some embodiments, the at least one divalent cation is not Mg²⁺. In some embodiments, the at least one divalent cation is not Ca²⁺. In some embodiments, the at least one divalent cation is not Sr²⁺. In some embodiments, the at least one divalent cation is not Ba²⁺. In some embodiments, the at least one divalent cation is not Mn²⁺. In some embodiments, the at least one divalent cation is not Co²⁺. In some embodiments, the at least one divalent cation is not Fe²⁺. In some embodiments, the at least one divalent cation is not Ni²⁺. In some embodiments, the at least one divalent cation is not Cu²⁺. In some embodiments, the at least one divalent cation is not Zn²⁺.

In some embodiments, a nucleic acid synthesis reaction comprising at least one divalent cation that is not Mg²⁺ proceeds at a faster rate than an identical reaction that comprises Mg²⁺ (either alone or in the presence of one or more other divalent cations).

In some embodiments, a nucleic acid synthesis reaction occurs under conditions in which magnesium is not present or is present in a concentration lower than a concentration of at least one other divalent cation.

In some embodiments, the nucleic acid synthesis reaction buffer comprises a pH buffering component. The buffering component is used at a concentration from 1 mM to 1M in the nucleic acid synthesis reaction buffer. In some embodiments, the buffering component is at a concentration of about 10 mM to about 100 mM. In some embodiments, the buffering component is at a concentration of about 100 to about 200 mM. In some embodiments, the buffering component is at a concentration of about 50 mM to about 100 mM. In some embodiments, the buffering component is at a concentration of about 10 mM to about 50 mM. In some embodiments, the buffering component is about a concentration of about 20 mM. Illustrative buffering components includes, without limitation, Tris (tris(hydroxymethyl)aminomethane), Tricine, bicine, Bis-Tris, CAPS, EPPS, HEPES (4-(2-hydroxyethyl)-1-piperazineethanesulfonic acid), MES, MOPS, PIPES, TAPS and TES. In some embodiments, the nucleic acid synthesis reaction buffer comprises Tris. In some embodiments, the nucleic acid synthesis reaction buffer comprises HEPES.

In some embodiments, the nucleic acid synthesis reaction buffer has a pH from about pH 6.0 to about pH 8.5. In some embodiments, the pH is about pH 6.0 to about pH 8.5. In some embodiments, the pH is about pH 6.5 to about pH 8.0. In some embodiments, the pH is about pH 7 to about pH 7.5. In some embodiments, the pH is about pH 7.5 to about pH 8.0. In some embodiments, the pH is about pH 8.

In some embodiments, the nucleic acid synthesis reaction buffer comprises a monovalent cation. The monovalent cation may be a salt. In some embodiments, the monovalent cation is selected from sodium, potassium, lithium, rubidium, cesium, ammonium or any combination thereof. In some embodiments, the monovalent cation is at a concentration of about 100 to about 200 mM

In some embodiments, the nucleic acid synthesis reaction buffer comprises a detergent, surfactant, or nonionic surfactant. In some embodiments, the detergent, surfactant, or nonionic surfactant is selected from TRITON X-100®, Nonidet P-40 (NP-40), Tween 20, P20, and Brij 35, or any combination thereof.

In some embodiments, the nucleic acid synthesis reaction buffer comprises one or more stabilizing agents. In some embodiments, the one or more stabilizing agents is bovine serum albumin and/or glycerol.

In some embodiments, the nucleic acid synthesis reaction buffer comprises one or more reducing agents. In some embodiments, the reducing agent is selected from dithiothreitol (DTT), tris (2-carboxyethyl) phosphine (TCEP), and β-mercaptoethanol.

### Formulating the Nucleic Acid Synthesis Reaction Buffer Composition

To prepare the nucleic acid synthesis reaction buffer compositions described herein, reagent components are mixed at working concentrations to form a solution suitable for immediate use with or without dilution or addition of further reagents. The water used in the formulations of the present disclosure can be distilled, deionized and sterile filtered (through a 0.1-0.2 micrometer filter), and is free of contamination by DNase and RNase enzymes. Such water is available commercially, for example from Sigma Chemical Company (Saint Louis, Mo.), or may be made as needed according to methods well known to those skilled in the art.

### Methods of Nucleic Acid Synthesis

Among other things, the present disclosure provides methods of nucleic acid synthesis. In some embodiments, a method of nucleic acid synthesis, comprising providing a conjugate reagent comprising a plurality of polymerase-nucleotide conjugates, wherein said conjugates comprise a nucleotide or a modified nucleotide covalently linked to a polymerase via a linker, and contacting a sample comprising a polynucleotide with said conjugate reagent, wherein said polymerase of the conjugate catalyzes an extension reaction comprising the covalent addition of a shielded nucleotide of the conjugate onto the 3′ hydroxyl of said polynucleotide in the nucleic acid synthesis reaction buffer disclosed herein.

In some embodiments, a method of nucleic acid synthesis, comprises contacting a polynucleotide with a polymerase and a nucleotide, wherein the effective concentration of the nucleotide relative to the polymerase has been increased artificially. In some embodiments, the method is carried out in the nucleic acid synthesis reaction buffer disclosed herein. By way of non-limiting example, an effective concentration of the nucleotide relative to the polymerase can be increased artificially by, for instance, engineering a polymerase to have greater affinity for the nucleotide to be incorporated by the polymerase in an extension reaction or tethering the nucleotide to the polymerase.

In some embodiments, the extension reaction has a faster turnover rate than an extension reaction performed in the presence of at least one divalent cation at a concentration greater than about 1 μM, about 2.5 μM, about 5 μM, about 10 μM, about 15 μM, about 20 μM, about 25 μM, about 30 μM, about 35 μM, about 40 μM, about 45 μM, about 50 μM, about 100 μM, about 150 μM, about 200 μM, about 250 μM, about 300 μM, about 350 μM, about 400 μM, about 450 μM, about 500 μM, about 1000 μM, about 1500 μM, about 2000 μM, about 2500 μM.

In some embodiments, the faster turnover rate is between about 1 second faster and about 60 seconds faster. In some embodiments, the faster turnover rate is about 1 second faster, about 2 seconds faster, about 3 seconds faster, about 4 seconds faster, about 5 seconds faster, about 6 seconds faster, about 7 seconds faster, about 8 seconds faster, about 9 seconds faster, about 10 seconds faster, about 15 seconds faster, about 20 seconds faster, about 25 seconds faster, about 30 seconds faster, about 35 seconds faster, about 40 seconds faster, about 45 seconds faster, about 50 seconds faster, about 55 seconds faster, or about 60 seconds faster.

In some embodiments, the faster turnover rate is between about 60 seconds faster and about 300 seconds faster. In some embodiments, the faster turnover rate is about 70 seconds faster. In some embodiments, the faster turnover rate is about 80 seconds faster. In some embodiments, the faster turnover rate is about 90 seconds faster. In some embodiments, the faster turnover rate is about 100 seconds faster. In some embodiments, the faster turnover rate is about 110 seconds faster. In some embodiments, the faster turnover rate is about 120 seconds faster. In some embodiments, the faster turnover rate is about 130 seconds faster. In some embodiments, the faster turnover rate is about 140 seconds faster. In some embodiments, the faster turnover rate is about 150 seconds faster. In some embodiments, the faster turnover rate is about 160 seconds faster. In some embodiments, the faster turnover rate is about 170 seconds faster. In some embodiments, the faster turnover rate is about 180 seconds faster. In some embodiments, the faster turnover rate is about 190 seconds faster. In some embodiments, the faster turnover rate is about 200 seconds faster. In some embodiments, the faster turnover rate is about 210 seconds faster. In some embodiments, the faster turnover rate is about 220 seconds faster. In some embodiments, the faster turnover rate is about 230 seconds faster. In some embodiments, the faster turnover rate is about 240 seconds faster. In some embodiments, the faster turnover rate is about 250 seconds faster. In some embodiments, the faster turnover rate is about 260 seconds faster. In some embodiments, the faster turnover rate is about 270 seconds faster. In some embodiments, the faster turnover rate is about 280 seconds faster. In some embodiments, the faster turnover rate is about 290 seconds faster. In some embodiments, the faster turnover rate is about 300 seconds faster.

### Determination of Turnover Rate

The methods of nucleic acid synthesis provided herein comprise a faster turnover rate for an extension reaction comprising the nucleic acid synthesis reaction buffer described herein. The turnover rate refers to the time required to extend an oligonucleotide by at least one nucleotide.

Any suitable method known in the art can be used for determining the turnover rate. The turnover rate can be assessed by analyzing nucleic acid synthesis products over time following the initiation of an extension reaction (i.e. reaction time course). Analyses can be performed by, for example, capillary electrophoresis (CE) as previously demonstrated (Smith and Nelson. Curr Protoc Nucleic Acid Chem. Chapter 10: Unit 10.9. 2003; Durney et al. Anal Bioanal Chem. 407:6923-6938. 2015). CE can separate and report abundance of polynucleotide products with single nucleotide resolution. The relative abundance of each nucleic acid product generated by the methods of nucleic acid synthesis provided herein can be analyzed by CE. By comparing the abundance of the starting material (i.e. initial polynucleotide or oligonucleotide to which a nucleotide is being incorporated) and the expected polynucleotide product, it is possible to determine the extent to which the extension reaction is completed. The change over time of the starting material and extended species is indicative of the turnover rate, as described herein. This approach to determine turnover rate has been demonstrated previously (Palluk et al. Nat Biotech. 36(7):645-650. 2018). Alternatively, analysis of nucleic acid synthesis products can be performed using reverse-phase high-performance liquid chromatography (RP-HPLC) as described previously (Jensen and Davis. Biochemistry. 57(12):1821-1832. 2018).

CE and RP-HPLC may also be used to determine the purity of each species in a nucleic acid synthesis product by determining the area under the curve for peaks in the electropherograms and chromatograms for CE and RP-HPLC, respectively. Any suitable software package suitable for fitting curves to electropherograms and chromatograms and calculating area under the curve (AUC) may be used to determine the abundance of each polynucleotide product in a plurality of nucleotide products.

### Conjugates

Described herein are methods of nucleic acid synthesis using conjugates comprising a polymerase and a nucleotide, wherein the polymerase and the nucleotide are linked via a linker that comprises a cleavable linkage. The polymerase moiety of a conjugate can elongate a nucleic acid using its linked nucleotide (i.e., the polymerase can catalyze the attachment of a nucleotide to which it is joined onto a nucleic acid) and remains attached to the elongated nucleic acid via the linker until the linker is cleaved.

### Overview

When a conjugate comprising a polymerase and a nucleoside polyphosphate is incubated with a nucleic acid, it preferentially elongates the nucleic acid using its tethered nucleotide (as opposed to using the nucleotide of another conjugate molecule). As described above, the polymerase then remains attached to the nucleic acid via its tether to the added nucleotide until exposed to some stimulus that causes cleavage of the linkage to the added nucleotide. In this situation, further extensions by polymerase-nucleotide conjugates are hindered (i.e., the nucleotide is “shielded”) when: 1) the attached polymerase molecule hinders other conjugates from accessing the 3′ OH of the extended DNA molecule and 2), other nucleoside polyphosphates in the system are hindered from accessing the catalytic site of the polymerase that remains attached to the 3′ end of the extended nucleic acid. (The extent of shielding may be described as the extent to which both of these interactions are hindered.) To enable subsequent extensions, the linker tethering the incorporated nucleotide to the polymerase can be cleaved, releasing the polymerase from the nucleic acid and therefore re-exposing its 3′ OH group for subsequent elongation.

Methods for nucleic acid synthesis provided herein that employ the shielding effect to achieve termination comprise an extension step wherein a nucleic acid is exposed to conjugates preferentially in the absence of free (i.e. untethered) nucleoside polyphosphates, because the termination mechanism of shielding may not prevent their incorporation into the nucleic acid.

In some embodiments, termination of further elongation may be “complete”, meaning that after a nucleic acid molecule has been elongated by a conjugate, further elongations cannot occur during the reaction. In other embodiments, termination of further elongation may be “incomplete”, meaning that further elongations can occur during the reaction but at a substantially decreased rate compared to the initial elongation, e.g., 100 times slower, or 1000 times slower, or 10,000 times slower, or more. Conjugates that achieve incomplete termination may still be used to extend a nucleic acid by predominantly a single nucleotide (e.g., in methods for nucleic acid synthesis and sequencing) when the reaction is stopped after an appropriate amount of time. In some embodiments, the reagent containing the conjugate may additionally contain polymerases without tethered nucleoside polyphosphates.

Reagents based on conjugates employing the shielding effect to achieve termination preferentially only contain polymerase-nucleotide conjugates in which all polymerases remain folded in the active conformation. In some cases, if the polymerase moiety of a conjugate is unfolded, its tethered nucleoside polyphosphate may become more accessible to the polymerase moieties of other conjugate molecules. In these cases, the unshielded nucleotides may be more readily incorporated by other conjugate molecules, circumventing the termination mechanism.

Polymerase-nucleotide conjugates employing the shielding effect to achieve termination are preferentially only labeled with a single nucleoside polyphosphate moiety. Polymerase-nucleotide conjugates labeled with multiple nucleoside polyphosphates that can access the catalytic site can, in some cases, incorporate multiple nucleoside polyphosphates into the same nucleic acid. Additional tethered nucleotides may therefore lead to additional, undesired nucleotide incorporations into a nucleic acid during a reaction. Furthermore, only one tethered nucleoside polyphosphates can occupy the (buried) catalytic site of its polymerase at a time so the other tethered nucleoside polyphosphate(s) may have an increasing accessibility to the polymerase moieties of other conjugate molecules, as discussed below.

Polymerase-nucleotide conjugates employing the shielding effect to achieve termination preferentially comprise as short of a linker as possible that still enables the nucleoside polyphosphate to frequently access the catalytic site of its tethered polymerase molecule in a productive conformation, in order to enable fast incorporation of the nucleotide into a nucleic acid. Such conjugates may also preferentially employ an attachment position of the linker to the polymerase as close to the catalytic site as possible, enabling use of a shorter linker. The length of the linker will determine the maximum distance from the attachment point a tethered nucleoside polyphosphate or a tethered nucleic acid can reach. A smaller distance may lead to a reduced accessibility of the tethered moiety to other polymerase-nucleotide molecules, as discussed below. In some embodiments, linkers are approximately 24 and 28 Å long. Shorter linkers, e.g. with lengths of 8-15 Å may increase shielding; longer linkers, e.g. linkers longer than 50 Å, 70 Å or 100 Å, may reduce shielding. The shielding effect may be influenced by a combination of factors including, but not limited to, the structure of the polymerase, the length of the linker, the structure of the linker, the attachment position of the linker to the polymerase, the binding affinity of the nucleoside polyphosphate to the catalytic site of the polymerase, the binding affinity of the nucleic acid to the polymerase, the preferred conformation of the polymerase, and/or the preferred conformation of the linker.

One contribution to shielding can be steric effects that block the 3′ OH of a nucleic acid that has been elongated by a conjugate from reaching into the catalytic site of another conjugate's polymerase moiety. Steric effects may also hinder a tethered nucleoside polyphosphate from reaching into the catalytic site of another polymerase-nucleotide conjugate molecule due to clashes between the conjugates that would occur during such approaches. These steric effects may result in complete termination if they completely block productive interactions between the tethered nucleoside polyphosphate (or elongated nucleic acid) of one conjugate molecule with another conjugate molecule, or may result in incomplete termination if they only hinder such intermolecular interactions.

Another contribution to shielding arises from the binding affinity of the tethered nucleoside polyphosphate to the catalytic site of the polymerase. The tethered nucleoside polyphosphate of a conjugate will have a high effective concentration with respect to the catalytic site of its tethered polymerase so it may remain bound to that site much of the time. When the nucleoside polyphosphate is bound to the catalytic site of its tethered polymerase molecule, it is unavailable for incorporation by other polymerase molecules. Thus, tethering reduces the effective concentration of nucleoside polyphosphates available for intermolecular incorporation (i.e. incorporation catalyzed by a polymerase molecule to which the nucleotide is not tethered). This shielding effect can enhance termination by reducing the rate by which a nucleic acid is elongated using the nucleoside polyphosphate moiety of one conjugate molecule by the polymerase moiety of another conjugate molecule.

Another contribution to shielding arises from the binding affinity of the 3′ region of a nucleic acid molecule to the catalytic site of a polymerase molecule. After elongation by a conjugate, the nucleic acid is tethered to the conjugate via its 3′ terminal nucleotide and will have a high effective concentration with respect to the catalytic site of its tethered polymerase so it may remain bound to that site much of the time. When the nucleic acid is bound to the catalytic site of its tethered polymerase molecule, it is unavailable for elongation by other conjugate molecules. This effect can enhance termination by reducing the rate by which a nucleic acid that has been elongated by a first conjugate is further elongated by other conjugate molecules.

In some embodiments, the polymerase-nucleotide conjugates comprise additional moieties that sterically hinder the tethered nucleoside polyphosphate (or a tethered nucleic acid post-elongation) from approaching the catalytic sites of another conjugate molecule. Such moieties include polypeptides or protein domains that can be inserted into a loop of the polymerase, and those and other bulky molecules such as polymers that can be site-specifically ligated e.g. to an inserted unnatural amino acid or specific polypeptide tag.

In some embodiments, the linker is attached to the 5 position of pyrimidines or the 7 position of 7-deazapurines. In other embodiments, the linker may be attached to an exocyclic amine of a nucleobase, e.g. by N-alkylating the exocyclic amine of cytosine with a nitrobenzyl moiety as discussed herein. In other embodiments, the linker may be attached to any suitable atom of the nucleotide to form a conjugate, such as the phosphate, sugar, or base of the nucleotide, as will be apparent to those skilled in the art. In some embodiments, the linker is attached to the alpha-phosphate, sugar, or base of the nucleotide so that the polymerase remains attached to the nucleotide after addition to the 3′ end of an oligonucleotide. In some embodiments, the linker is attached to the β-phosphate, χ-phosphate, δ-phosphate, ε-phosphate, ϕ-phosphate, or γ-phosphate of a nucleotide. In some embodiments, the linker is attached to the terminal phosphate of a nucleotide

Certain polymerases have a high tolerance for modification of certain parts of a nucleotide, e.g. modifications of the 5-position of pyrimidines and the 7-position of purines are well-tolerated by some polymerases (He and Seela., Nucleic Acids Research 30.24 (2002): 5485-5496.; or Hottin et al., Chemistry. 2017 Feb. 10; 23(9):2109-2118). In some embodiments, the linker is attached to these positions.

In some embodiments, a polymerase-nucleotide conjugate is prepared by first synthesizing an intermediate compound comprising a linker and a nucleotide (referred to herein as a “linker-nucleotide”), and then the intermediate compound is attached to the polymerase. By way of non-limiting examples, in some embodiments, nucleosides with substitutions compared to natural nucleosides, e.g. pyrimidines with 5-hydroxymethyl or 5-propargylamino substituents, or 7-deazapurines with 7-hydroxymethyl or 7-propargylamino substituents may be useful starting materials for preparing linker-nucleotides. An exemplary set of nucleosides with 5- and 7-hydroxymethyl substituents that may be useful for preparing linker-nucleotides is shown below:

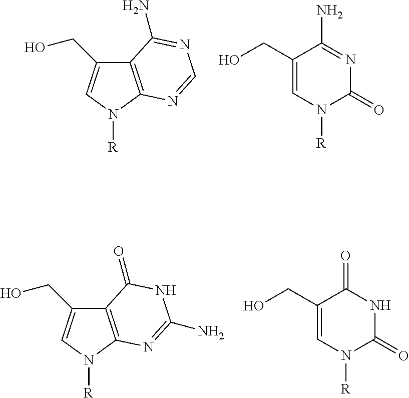

An exemplary set of nucleosides with 5- and 7-deaza-7-propargylamino substituents that may be useful for preparing linker-nucleotides is shown below:

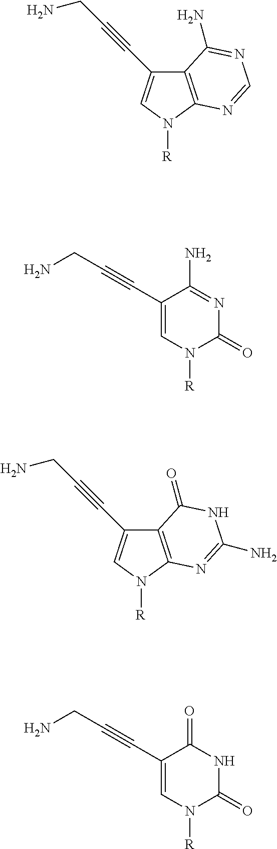

These nucleosides are also commercially available as deoxyribonucleoside polyphosphates.

In some embodiments a method of preparation (e.g., comprising an intermediate compound), the conjugate comprises a linker-nucleotide.

In some embodiments, the linker-nucleotide comprises a nucleotide. In some embodiments, the linker-nucleotide comprises a nucleotide polyphosphate or a modified nucleotide polyphosphate. In some embodiments, the linker-nucleotide comprises a nucleotide triphosphate or a modified nucleotide triphosphate. Any suitable nucleotide may be used. It is understood that a nucleotide comprises a nucleobase (e.g. adenine, guanine, cytosine thymine or uracil), a sugar (e.g. a ribose or a deoxyribose), and a polyphosphate. It is understood that a nucleoside comprises a nucleobase (e.g. adenine, guanine, cytosine thymine or uracil) and a sugar (e.g. a ribose or a deoxyribose).

In some embodiments, the linker-nucleotide comprises a nucleotide polyphosphate. In some embodiments, the linker-nucleotide comprises a modified nucleotide polyphosphate. It is understood that the polyphosphate portion of a nucleotide can be a monophosphate, a diphosphate, a triphosphate, a tetraphosphate, a heptaphosphate, or a pentaphosphate. In some embodiments, the nucleotide polyphosphate comprises a nucleoside triphosphate or a modified nucleoside triphosphate. In some embodiments, the linker-nucleotide comprises a nucleotide tetraphosphate or a modified nucleotide tetraphosphate. In some embodiments, the linker-nucleotide comprises a nucleotide pentaphosphate or a modified nucleotide pentaphosphate. In some embodiments, the linker-nucleotide comprises a nucleotide hexaphosphate or a modified nucleotide hexaphosphate.

In some embodiments, the linker-nucleotide comprises a modified nucleobase. In some embodiments, the linker-nucleotide comprises a modified nucleobase. In some embodiments, the modified nucleobase comprises an O- or N-linked modification. In some embodiments, the O- or N-linked modification is removable following incorporation of the nucleotide portion of the linker-nucleotide into a polynucleotide. In some embodiments, the O- or N-linked modification is removable by a photolytic process. In some embodiments, the photolytic process comprises exposure to UV light, wherein the UV light comprises wavelengths at 365 nm and/or 405 nm. In some embodiments, the O- or N-linked modification is removable by a chemical process. In some embodiments, the chemical process is selected from a beta-elimination reaction, a Pd-catalyzed deallylation, and a reduction reaction. In some embodiments, the O- or N-linked modification is removable by an enzymatic process. In some embodiments, the enzymatic process comprises removal by an alkyltransferase or methyltransferase.

In some embodiments, the O- or N-linked modification reduces or eliminates Watson-Crick base pairing in a polynucleotide comprising the modified nucleobase. In some embodiments, the O- or N-linked modification reduces or eliminates secondary structure in a polynucleotide comprising the modified nucleobase. In some embodiments of the method, following removal of the O- or N-linked modification the modified nucleobase comprises a natural nucleobase. In some embodiments, the natural nucleobase is guanine, cytosine, adenine, thymine, or uracil.

### Linker-Polymerase Attachment

The conjugates provided herein comprise a polymerase tethered to a nucleotide via a linker.

Any suitable linker for tethering a nucleoside polyphosphate to a polymerase is contemplated for use in the methods described herein. In some embodiments, the linker is specifically attached to a cysteine residue of the polymerase using a sulfhydryl-specific attachment chemistry. Illustrative sulfhydryl specific attachment chemistries include, without limitation, ortho-pyridyl disulfide (OPSS), maleimide functionalities, 3-arylpropiolonitrile functionalities, allenamide functionalities, haloacetyl functionalities such as iodoacetyl or bromoacetyl, alkyl halides or perfluroaryl groups that can favorably react with sulfhydryls surrounded by a specific amino acid sequence (Zhang, Chi, et al. Nature chemistry 8, (2015) 120-128.). Other attachment chemistries for specific labeling of cysteine residues will be apparent to those skilled in the art or are described in the pertinent literature and texts (e.g., Kim, Younggyu, et al, Bioconjugate chemistry 19.3 (2008): 786-791.).

In some embodiments, the linker is attached to a lysine residue via an amine-reactive functionality (e.g. NHS esters, Sulfo-NHS esters, tetra- or pentafluorophenyl esters, isothiocyanates, sulfonyl chlorides, etc.). In some embodiments, the linker is attached to the polymerase via attachment to a genetically inserted unnatural amino acid, e.g. p-propargyloxyphenylalanine or p-azidophenylalanine that could undergo azide-alkyne Huisgen cycloaddition, though many suitable unnatural amino acids suitable for site-specific labeling exist and can be found in the literature (e.g. as described in Lang and Chin., Chemical reviews 114.9 (2014): 4764-4806.).

In some embodiments, the linker may be specifically attached to the polymerase N-terminus. In some embodiments, the polymerase is mutated to have an N-terminal serine or threonine residue, which may be specifically oxidized to generate an N-terminal aldehyde for subsequent coupling to e.g. a hydrazide. In some embodiments, the polymerase is mutated to have an N-terminal cysteine residue that can be specifically labeled with an aldehyde to form a thiazolidine. In some embodiments, an N-terminal cysteine residue can be labeled with a peptide linker via Native Chemical Ligation.

In some embodiments, a peptide tag sequence may be inserted into the polymerase that can be specifically labeled with a synthetic group by an enzyme, e.g. as demonstrated in the literature using biotin ligase, transglutaminase, lipoic acid ligase, bacterial sortase and phosphopantetheinyl transferase (e.g. as described in refs. 74-78 of Stephanopoulos & Francis Nat. Chem. Biol. 7, (2011) 876-884).

In some embodiments, the linker is attached to a labeling domain fused to the polymerase. For example, a linker with a corresponding reactive moiety may be used to covalently label SNAP tags, CLIP tags, HaloTags and acyl carrier protein domains (e.g. as described in refs. 79-82 of Stephanopoulos & Francis Nat. Chem. Biol. 7, (2011) 876-884).

In some embodiments, the linker is attached to an aldehyde specifically generated within the polymerase, as described in Carrico et al. (Nat. Chem. Biol. 3, (2007) 321-322). For example, after insertion of an amino acid sequence that is recognized by the enzyme formylglycine-generating enzyme (FGE) into the polymerase, it may be exposed to FGE, which will specifically convert a cysteine residue in the recognition sequence to formylglycine (i.e. producing an aldehyde). This aldehyde may then be specifically labeled with e.g. a hydrazide or aminooxy moiety of a linker.

In some embodiments, a linker may be attached to the polymerase via non-covalent binding of a moiety of the linker to a moiety fused to the polymerase. Examples of such attachment strategies include fusing a polymerase to streptavidin that can bind a biotin moiety of a linker, or fusing a polymerase to anti-digoxigenin that can bind a digoxigenin moiety of a linker. In some embodiments, site-specific labeling may lead to an attachment of the linker to the polymerase that may readily be reversed (e.g. an ortho-pyridyl disulfide (OPSS) group that forms a disulfide bond with a cysteine that can be cleaved using reducing agents, e.g. using TCEP), other attachment chemistries will produce permanent attachments.

In some embodiments, the polymerase is mutated to ensure specific attachment of the tethered nucleotide to a particular location of the polymerase, as will be apparent to those skilled in the art. For example, with sulfhydryl-specific attachment chemistries such as maleimides or ortho-pyridyl disulfides, accessible cysteine residues in the wild-type polymerase may be mutated to a non-cysteine residue to prevent labeling at those positions. On this “reactive cysteine-free” background, a cysteine residue may be introduced by mutation at the desired attachment position. These mutations preferentially do not interfere with the activity of the polymerase.

Other strategies for site-specific attachment of synthetic groups to proteins will be apparent to those skilled in the art and are reviewed in literature, (e.g. Stephanopoulos & Francis Nat. Chem. Biol. 7, (2011) 876-884).

### Polymerases

As described above, when a conjugate comprising a polymerase (e.g., a template-independent polymerase) and a nucleotide is incubated with a nucleic acid or a polynucleotide, it preferentially elongates (i.e. extends) the nucleic acid or polynucleotide by incorporating the tethered nucleotide or modified nucleotide (as opposed to using the nucleotide or modified nucleotide of another conjugate molecule) into the nucleic acid or polynucleotide. In some embodiments, a polymerase in a polymerase-nucleotide conjugate is folded in an active conformation. In other embodiments, a polymerase in a polymerase-nucleotide conjugate is unfolded.

Any polymerase capable of extending a polynucleotide, incorporating a nucleotide into a polynucleotide, or incorporating a nucleotide analog into a polynucleotide is envisaged for use in the methods described herein. In some embodiments, the polynucleotide is single stranded. In some embodiments, the polynucleotide is double stranded. In some embodiments, the polynucleotide is immobilized on a solid support.

For DNA synthesis applications, in particular template-independent polymerases, e.g., a terminal deoxynucleotidyl transferase (TdT) or DNA nucleotidylexotransferase, which terms are used interchangeably to refer to an enzyme having activity as described for E.C. class 2.7.7.31 may be used.

In some embodiments, methods of the present disclosure use conjugates comprising template-independent polymerases. In some embodiments, conjugates comprise a Pol-X family polymerase. In some embodiments, conjugates comprise a polymerase Terminal deoxynucleotidyl Transferase (TdT), or a variant thereof (e.g., a non-wild-type TdT, e.g., a modified TdT). In some embodiments of the method, the template-independent polymerase is a TdT or a variant thereof (i.e., a modified TdT). In some embodiments of the method, the TdT or variant thereof comprises a sequence sharing at least 60%, at least 65%, at least 70%, at least 75%, at least 80%, at least 85%, at least 90%, at least 95%, at least 96%, at least 97%, at least 98%, or at least 99% identity to SEQ ID NO: 1 or a portion thereof. For example, in some embodiments, the TdT comprises a sequence identical to a portion of a particular TdT (e.g., that of SEQ ID NO: 1, e.g., that of SEQ ID NO: 1, etc.). For instance, in some embodiments a given TdT may be truncated relative to the length of a particular TdT such as that set forth in SEQ ID NO: 1. In some embodiments, a TdT may be a circular permutation of SEQ ID NO: 1). In some embodiments, a TdT variant comprises one or more amino acid substitutions, insertions, or deletions., and/or is a circular permutant thereof relative to a reference TdT (e.g., a wild-type TdT, a modified TdT, etc.).

In some embodiments of the method, the polymerase is a fusion protein. In some embodiments of the method, the fusion protein comprises maltose binding protein (MBP).

In some embodiments of the method, the TdT or variant thereof may be operably linked to a linker moiety including a covalent or non-covalent bond; amino acid tag (e.g., poly-amino acid tag, poly-His tag, 6His-tag (SEQ ID NO: 2)); chemical compound (e.g., polyethylene glycol); protein-protein binding pair (e.g., biotin-avidin); affinity coupling; capture probes; or any combination of these. The linker moiety can be separate from or part of a TdT variant.

>Terminal deoxynucleotidyl transferase (TdT)
(SEQ ID NO: 1)
MGGRDIVDGSEFSPSPVPGSQNVPAPAVKKISQYACQRRTTLNNYNQLF
TDALDILAENDELRENEGSALAFMRASSVLKSLPFPITSMKDTEGIPSL
GDKVKSIIEGIIEDGESSEAKAVLNDERYKSFKLFTSVFGVGLKTAEKW
FRMGFRTLSKIQSDKSLRFTQMQKAGFLYYEDLVSCVNRPEAEAVSMLV
KEAVVTFLPDALVTMTGGFRRGKMTGHDVDFLITSPEATEDEEQQLLHK
VTDFWKQQGLLLYADILESTFEKFKQPSRKVDALDHFQKCFLILKLDHG
RVHSEKSGQQEGKGWKAIRVDLVMSPYDRRAFALLGWTGSRQFERDLRR
YATHERKMMLDNHALYDRTKRVELEAESEEEIFAHLGLDYIEPWERNA

Illustrative examples of polymerases with the ability to extend single stranded nucleic acids include, but are not limited to, Polymerase Theta (Kent et al., eLife 5 (2016): el3740.), polymerase mu (Juarez et al., Nucleic acids research 34.16 (2006): 4572-4582; or McElhinny et all., Molecular cell 19.3 (2005): 357-366.) or polymerases where template-independent activity is induced, e.g. by the insertion of elements of a template-independent polymerase (Juarez et al., Nucleic acids research 34.16 (2006): 4572-4582). In other DNA synthesis applications, the polymerase can be a template-dependent polymerase i.e., a DNA-directed DNA polymerase (which terms are used interchangeably to refer to an enzyme having activity 2.7.7.7 using the IUBMB nomenclature).

In some embodiments, such as RNA synthesis applications, tethered ribonucleotides (e.g., ribonucleostide polyphosphates) may be used. In some such embodiments, a RNA specific nucleotidyl transferase, such as E. coli Poly(A) Polymerase (IUBMB EC 2.7.7.19) or Poly(U) Polymerase, among others, may be employed. The RNA nucleotidyl transferases can contain modifications, e.g., single point mutations, that influence the substrate specificity towards a specific rNTP (Lunde et al., Nucleic acids research 40.19 (2012): 9815-9824.). In some embodiments, a very short tether between an RNA nucleotidyl transferase and a ribonucleotide (e.g., ribonucleoside triphosphate) may be used to induce a high effective concentration of the ribonucleotide (e.g., ribonucleoside polyphosphate), thereby forcing incorporation of an rNTP that might not be the natural substrate of the nucleotidyl transferase.

### Linker

In some embodiments, a conjugate of the present disclosure comprises a linker. In some such embodiments, the linker comprises at least the atoms that connect the nucleotide to the polymerase. The linker can attach to the base, the sugar, or the aphosphate of the nucleotide or modified nucleotide to the polymerase. In some embodiments, the polymerase and the nucleotide are attached with a linker. In some such embodiments, the polymerase and the nucleotide are covalently linked (via the linker) and the distance between the linked atom of the nucleotide and the polymerase to which it is attached can be, for example, in the range of about 4-100 Å, about 15-40 Å or about 20-30 Å, or a distance appropriate for the position on the polymerase to which the nucleotide (e.g., nucleoside polyphosphate) is tethered. Any suitable linker for tethering the nucleotide or modified nucleotide to the polymerase is contemplated in the methods described herein. In some embodiments, the linker comprises a polyether or a polyethylene glycol (PEG). In some embodiments, the linker comprises one or more peptide bonds. In some embodiments, the linker comprises one or more sarcosines. In some embodiments, the linker comprises one or more glycines. In some embodiments, the linker comprises one or more prolines. In some embodiments, the linker comprises a carbamate. In some embodiments, the linker joins to the nucleotide at an atom of the nucleobase that is not involved in base pairing. In such embodiments, the linker is considered to be at least the atoms that connect the polymerase to any atom in the monocyclic or polycyclic ring system bonded to the Γ position of the sugar (e.g. pyrimidine or purine or 7-deazapurine or 8-aza-7-deazapurine). In some embodiments, the linker joins to the nucleotide at an atom of the nucleobase that is involved in base pairing. In some embodiments, the linker is joined to the sugar or to the α-phosphate of the nucleotide. In some embodiments, the linker is sufficiently long to allow the nucleotide (e.g., nucleoside polyphosphate) to access the active site of the polymerase to which it is tethered. As described in greater detail herein, the polymerase of a conjugate is capable of catalyzing the addition of the nucleotide to which it is linked onto the 3′ end of a nucleic acid.

### Cleavage of Linker

As described herein, a the linker may be attached to various positions on a nucleotide (e.g., of a conjugate of the present disclosure), and a variety of cleavage strategies may be used. It is understood that the cleavage strategy will be determined by the type of linker joining the nucleotide or modified nucleotide and the polymerase. Any suitable method for cleaving a linker is contemplated in the methods described herein.

In some embodiments, the linker is cleaved, wherein following cleavage of the linker, a nucleotide comprising a chemical group from the retained portion of the linker (i.e. a scar) is formed. e.g. illustrative, non-limiting, chemical groups (i.e. scars) following linker cleavage are shown below. In some embodiments, the chemical group e.g.is removed by a chemical, photolytic, or enzymatic process.

In some embodiments, the linker may be cleaved by exposure to any suitable reducing agent such as dithiothreitol (DTT), β-mercaptoethanol, or tris(2-carboxyethyl) phosphine (TCEP). For example, a linker comprising a 4-(disulfaneyl) butanoyloxy-methyl group attached to the 5 position of a pyrimidine or the 7 position of a 7-deazapurine may be cleaved by reducing agents (e.g. DTT) to produce a 4-mercaptobutanoyloxymethyl scar on the nucleobase. This scar may undergo intramolecular thiolactonization to eliminate a 2-oxothiolane, leaving a smaller hydroxymethyl scar on the nucleobase. An example of such a linker attached to the 5-position of cytosine is depicted below, but the strategy is applicable to any suitable nucleobase:

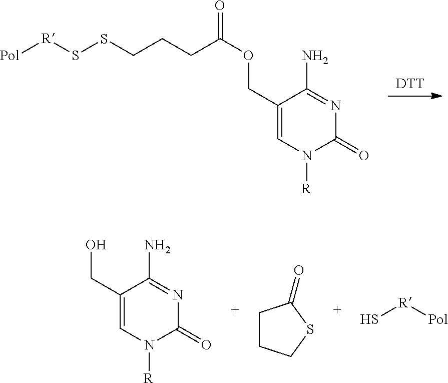

In other embodiments, the linker may be cleaved by exposure to light. For example a linker comprising a (2-nitrobenzyl)oxymethyl group may be cleaved with 365 nm light, leaving a hydroxymethyl scar, e.g. as depicted for cytosine below, but the strategy is applicable to any suitable nucleobase:

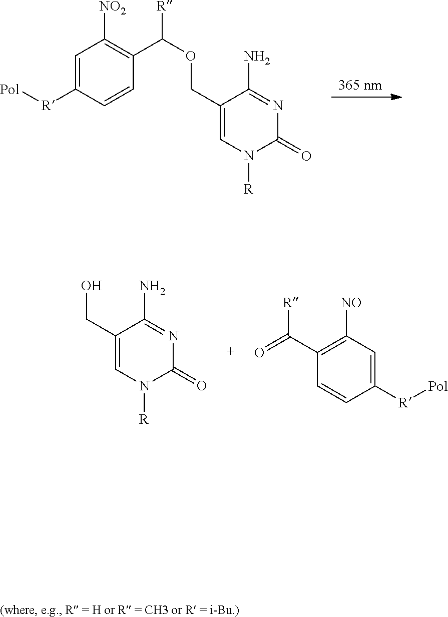

In other embodiments, the linker may comprise a 3-(((2-nitrobenzyl)oxy)carbonyl)aminopropynyl group that may be cleaved with 365 nm light to release a nucleobase with a propargylamino scar. This strategy is applicable to any suitable nucleobase:

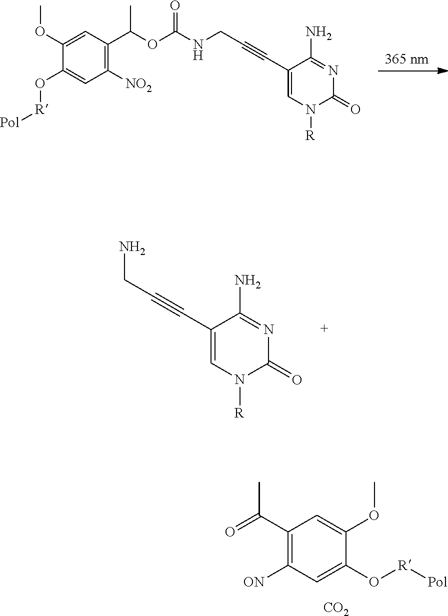

In other embodiments, the linker may comprise an acyloxymethyl group that may be cleaved with a suitable esterase to release a nucleobase with a hydroxymethyl scar, e.g. as depicted for cytosine below, but the strategy is applicable to any suitable nucleobase:

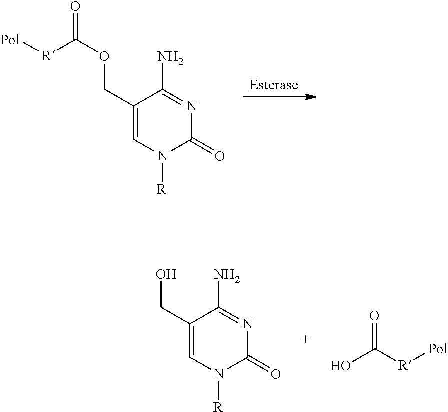

In such embodiments, the linker may comprise additional atoms (included in R′ above) adjacent to the ester that increase the activity of the esterase towards the ester bond.

In other embodiments, the linker may comprise an N-acyl-aminopropynyl group that may be cleaved with a peptidase to release a nucleobase with propargylamino scar, e.g. as depicted for 5-propargylamino cytosine below, but the strategy is applicable to any suitable nucleobase:

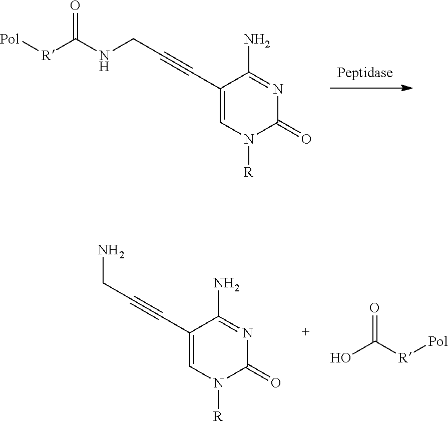

In such embodiments, the linker may comprise additional atoms (included in R′ above) adjacent to the amide that increase the activity of the peptidase towards the amide bond.

### EQUIVALENTS AND SCOPE

Those skilled in the art will recognize, or be able to ascertain using no more than routine experimentation, many equivalents to the specific embodiments in accordance with the compositions and methods described herein. The scope of the present disclosure is not intended to be limited to the disclosure herein but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

It is also noted that the term “comprising” is intended to be open and permits, but does not require, the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The term “about” as used herein refers to the normal range of error for each value readily known to those skilled in the art. The term “about” value or reference to a parameter herein includes (and describes) an implementation of the value or parameter itself. For example, a description referring to “about X” includes a description of “X”. In some embodiments, “about” means a value of at most +/−10% of the recited value, e.g., +/−1%, +/−2%, +/−3%, +/−4%, +/−5%, +/−6% %, +8%, +9%, or =10%.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the embodiments of the present disclosure may employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B(1992).

### Example 1: Impact of Cobalt Concentrations on the Rate of Nucleotide Incorporation by a DNA Polymerase-Nucleotide Conjugate

### Reduced Divalent Metal Concentration Increases Rate of Nucleotide Incorporation

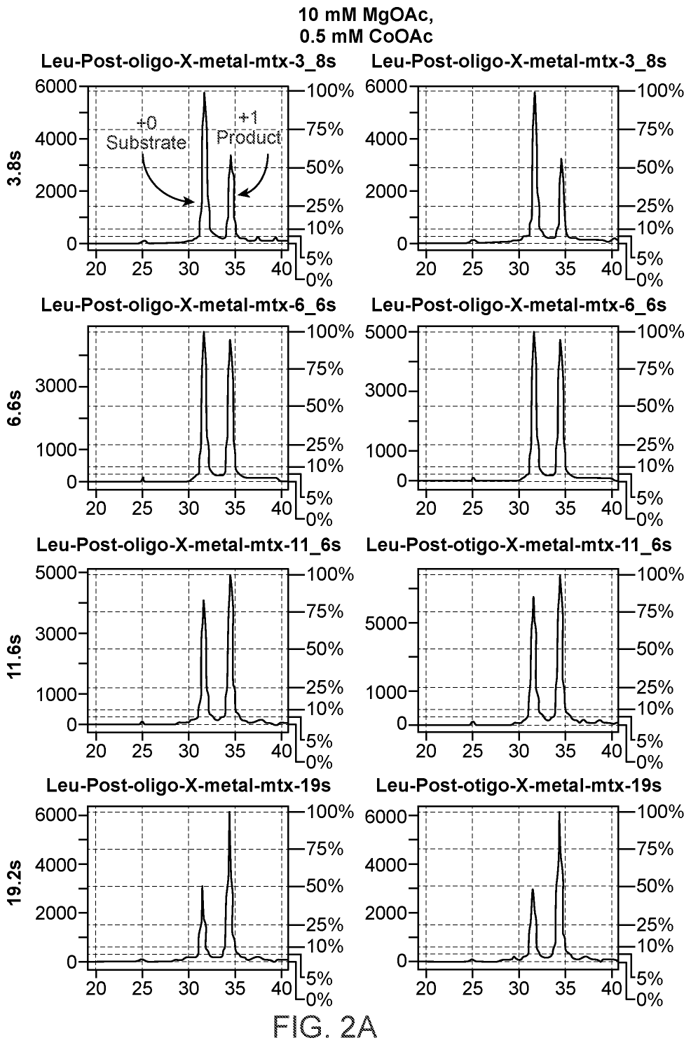

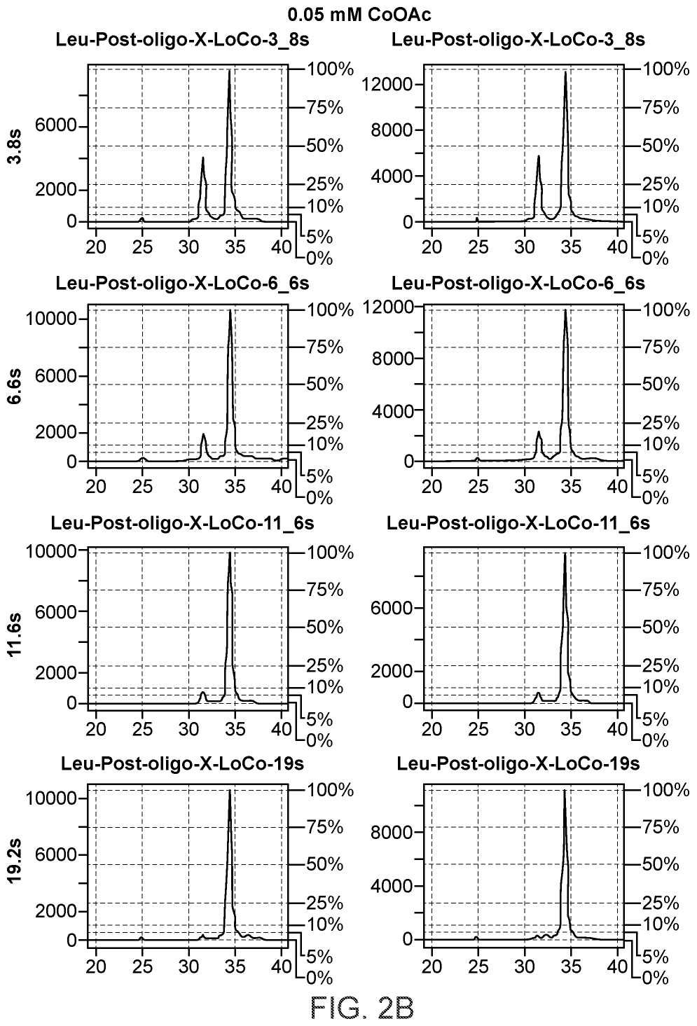

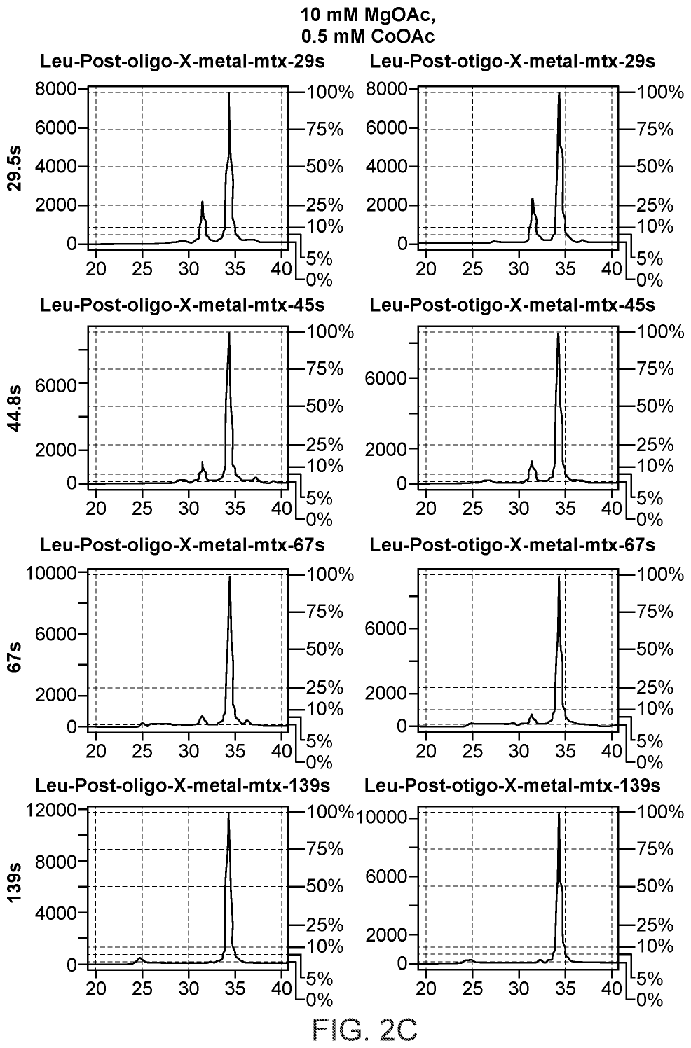

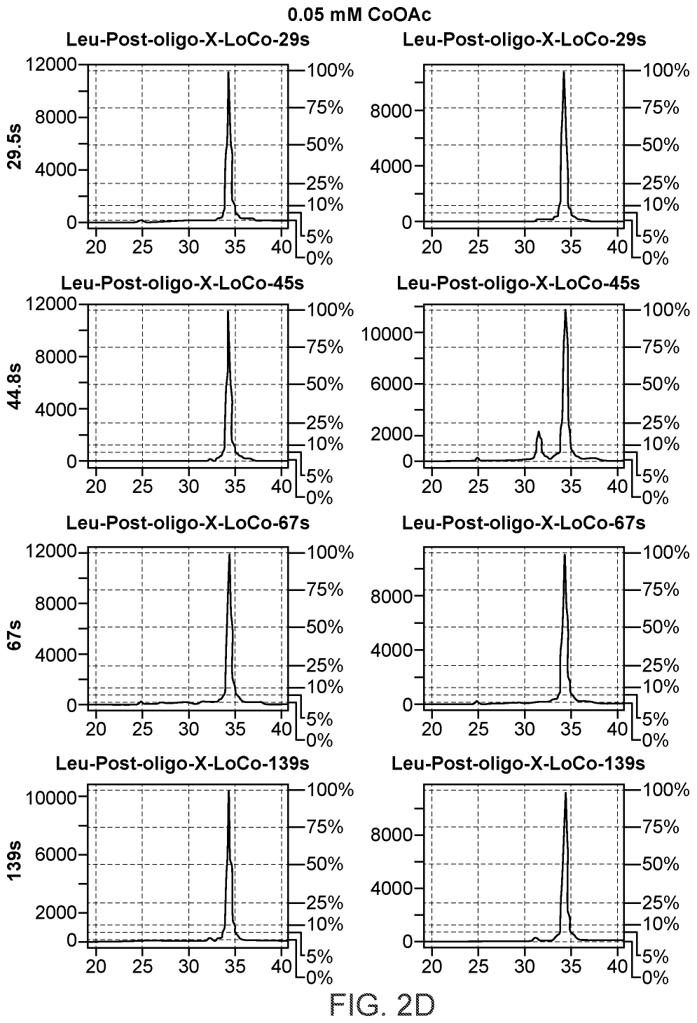

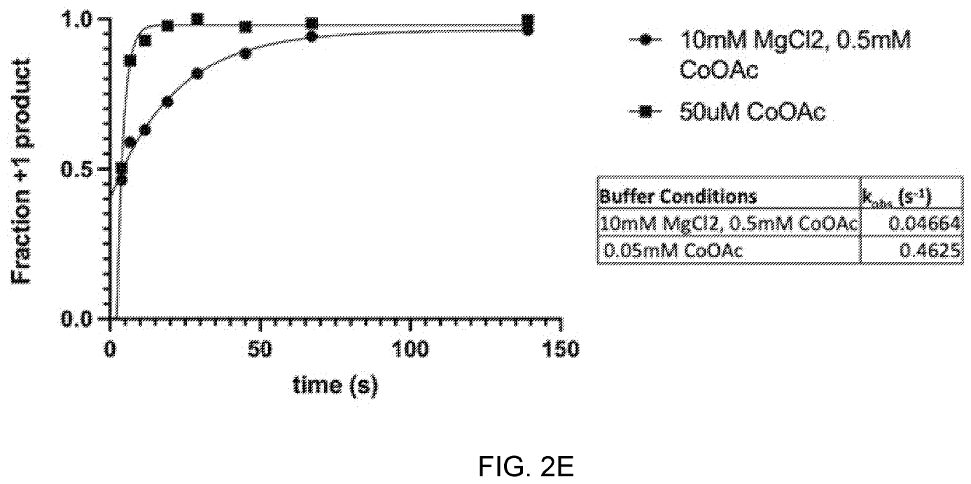

In this example, the nucleotide incorporation rates were analyzed for a TdT enzyme conjugated to a G-nucleotide using standard magnesium acetate and cobalt acetate concentrations and compared to a low cobalt acetate concentration (see FIGS. 2A-2E).

Enzymatic polynucleotide synthesis was carried out in buffer containing 20 mM Tris Acetate, 50 mM Potassium Acetate, 50 μM (FIGS. 2B and 2D) or 0.5 mM cobalt acetate (FIGS. 2A and 2C), 0 mM (FIGS. 2B and 2D) or 10 mM (FIGS. 2A and 2C) magnesium acetate, 50 nM DNA oligo substrate, 1 μM TdT enzyme-nucleotide conjugate, pH 7.9. Polymerase-nucleotide conjugate kinetic activity was measured upon quenching reactions with EDTA and performing DNA fragment analysis by capillary electrophoresis.

FIGS. 2A-2D show abundance of the DNA oligo substrate (substrate) and the product containing the incorporated nucleotide (product) at various time points in a polynucleotide extension reaction including TdT enzyme conjugated to a G-nucleotide in reaction buffer containing a standard concentration of: (i) magnesium acetate (10 mM MgOAc) and cobalt acetate (0.5 mM CoOAc) (see FIGS. 2A and 2C); or (ii) low cobalt acetate (0.05 mM CoOAc) (see FIGS. 2B and 2D) in the absence of magnesium acetate. Surprisingly, extension reactions containing no magnesium acetate and low cobalt acetate conditions yields complete incorporation of a nucleotide into polynucleotide after 30 seconds. In contrast, extension reactions containing the standard magnesium acetate and cobalt acetate concentrations were still incomplete after 67 seconds.

Results from synthesis/extension reactions shown in FIGS. 2A and 2B were quantified (over all time points) and reaction rates were determined for low cobalt and high cobalt buffer conditions (FIG. 2E). The calculated reaction rate (k_{obs}) for the low cobalt acetate condition (0.05 mM CoOAc, k_{obs}=0.4625) was an order of magnitude greater than the calculated reaction rate for the high cobalt acetate condition (10 mM MgCl2, 0.5 mM CoOAc, k_{obs}=0.04664).

These results indicate that compared to standard concentrations of cobalt acetate and magnesium acetate, a low cobalt acetate concentration in the absence of magnesium promotes a faster nucleotide incorporation rate for polynucleotide extension reactions using a DNA polymerase-nucleotide conjugate.

Rate of Nucleotide Incorporation Increases with Reducing Divalent Metal Concentration

In this example, the nucleotide incorporation rates were analyzed for a TdT enzyme conjugated to a G-nucleotide using a range of cobalt acetate or zinc acetate concentrations in the absence of magnesium acetate.

Enzymatic polynucleotide synthesis was carried out in buffer containing 20 mM Tris Acetate, 50 mM Potassium Acetate, 50 μM-2.5 mM cobalt acetate or 50 μM-2.5 mM zinc acetate, 50 nM DNA oligo substrate, 1 μM TdT enzyme-nucleotide conjugate, pH 7.9. Polymerase-nucleotide conjugate kinetic activity was measured upon quenching reactions with EDTA and performing DNA fragment analysis by capillary electrophoresis.

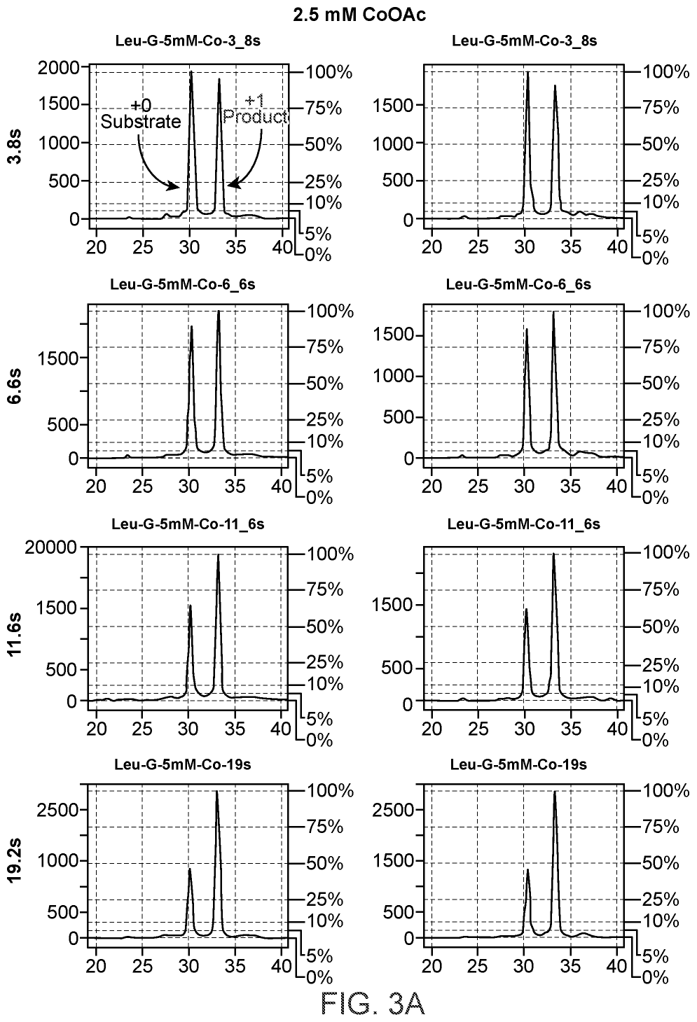

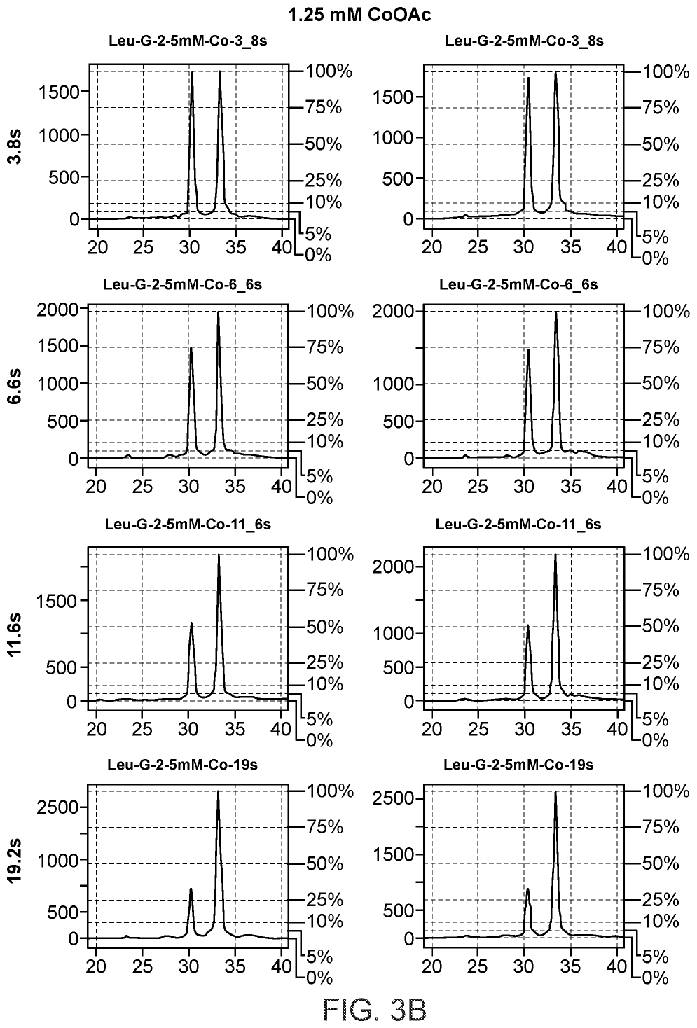

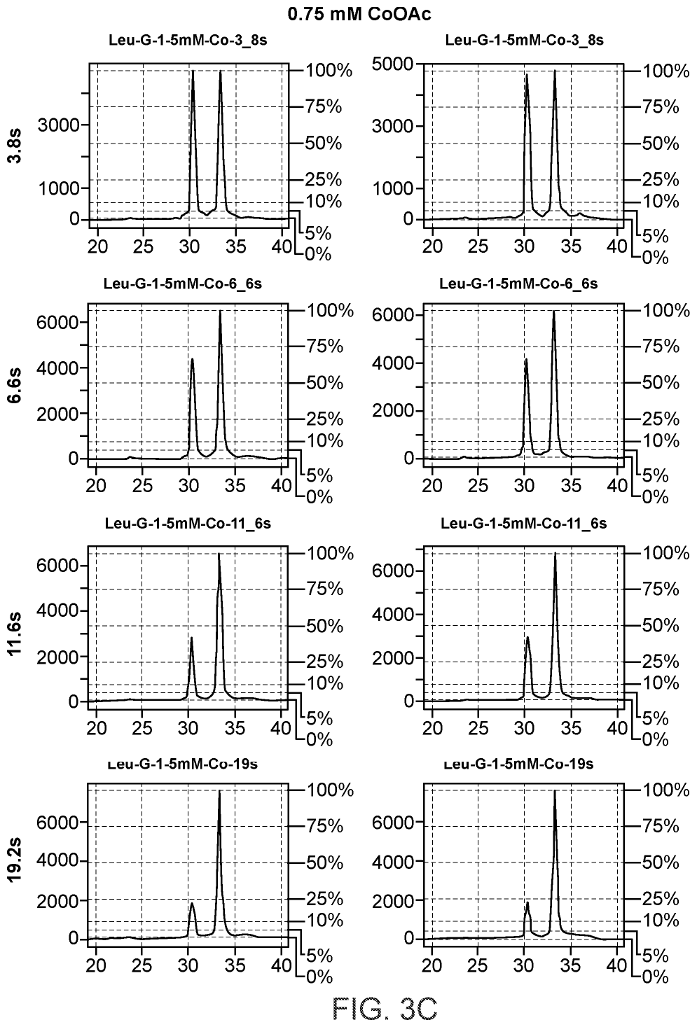

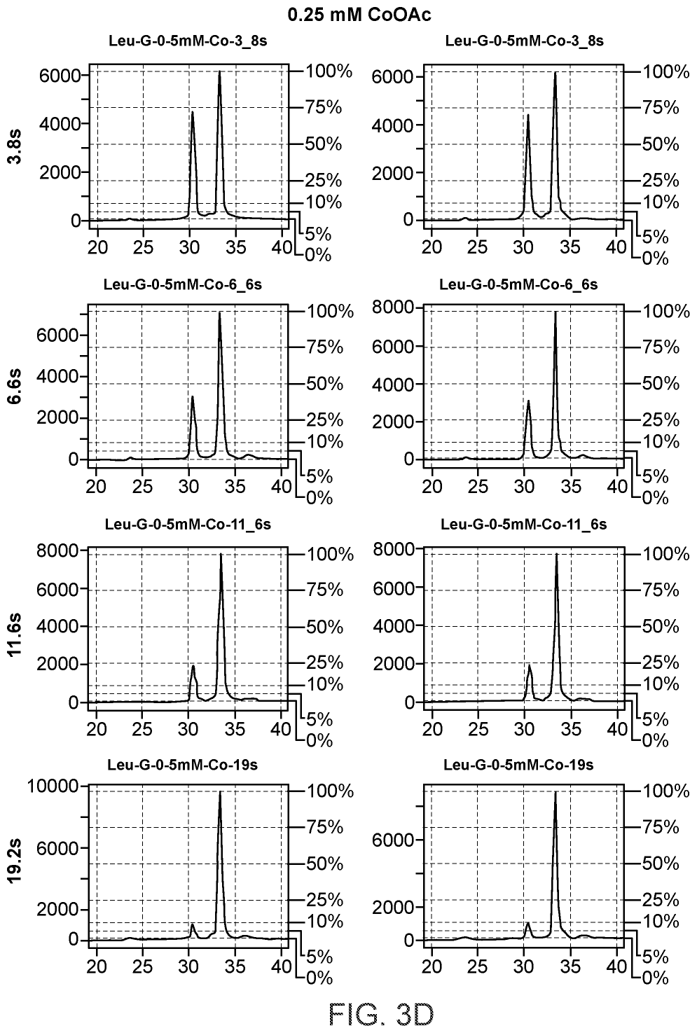

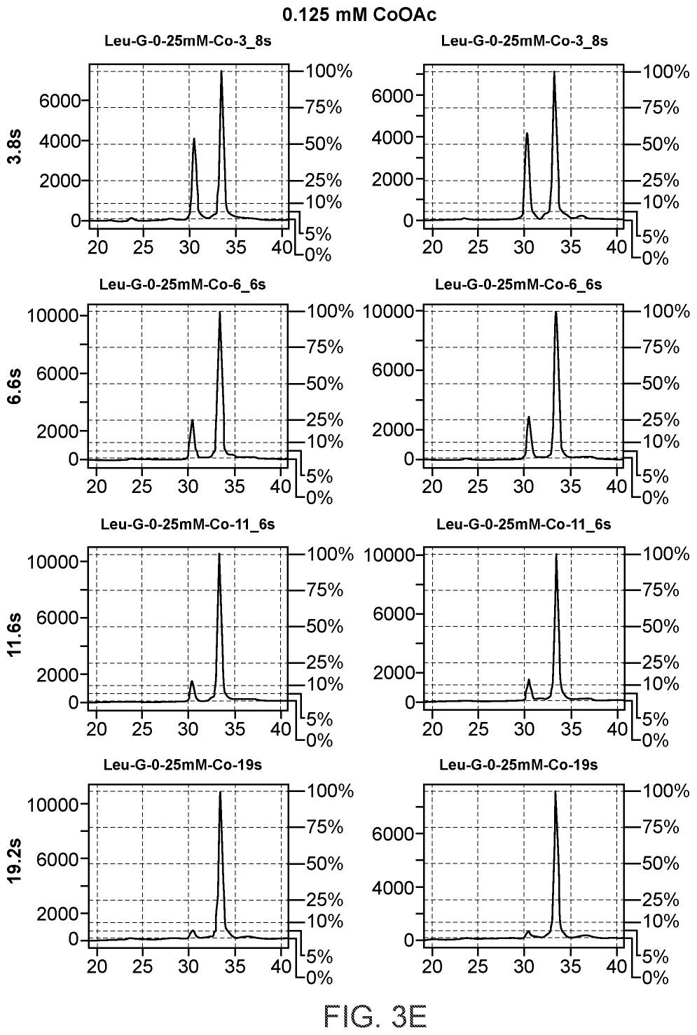

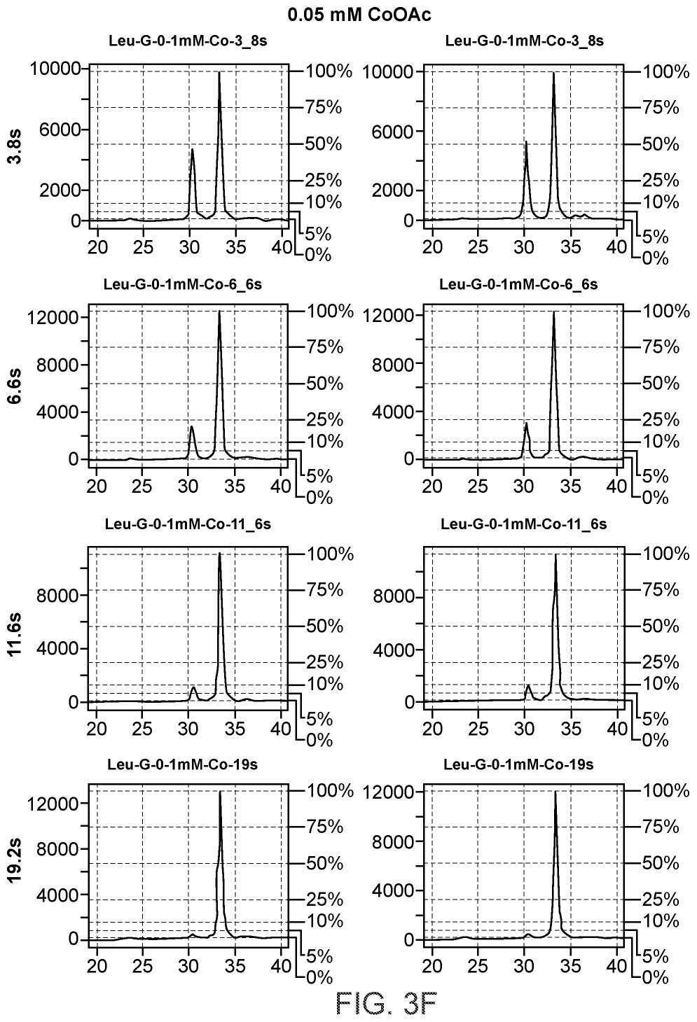

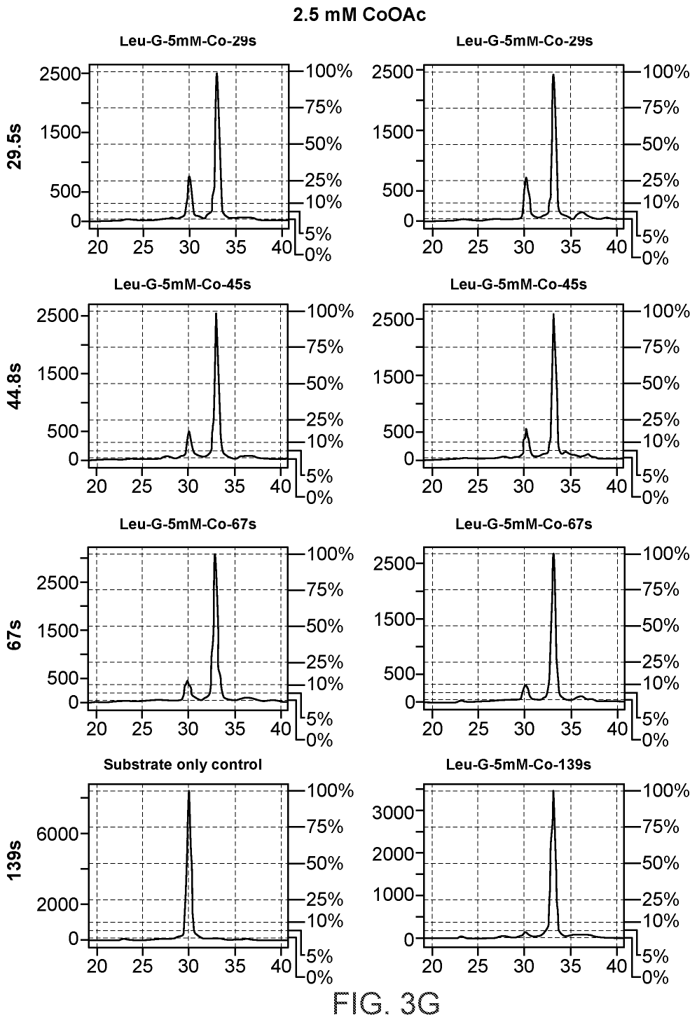

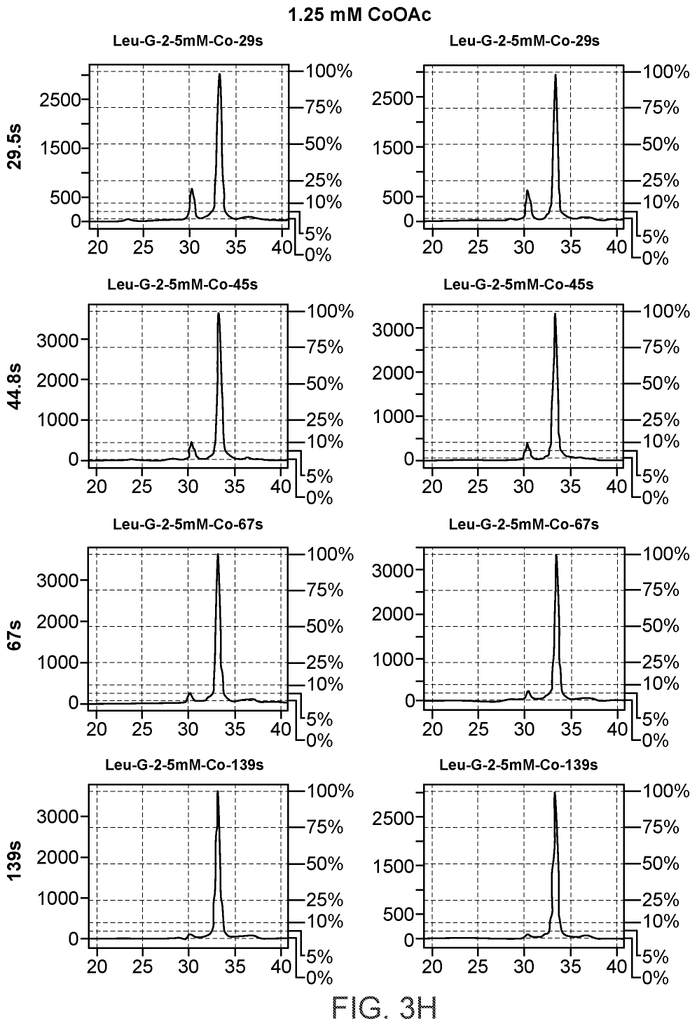

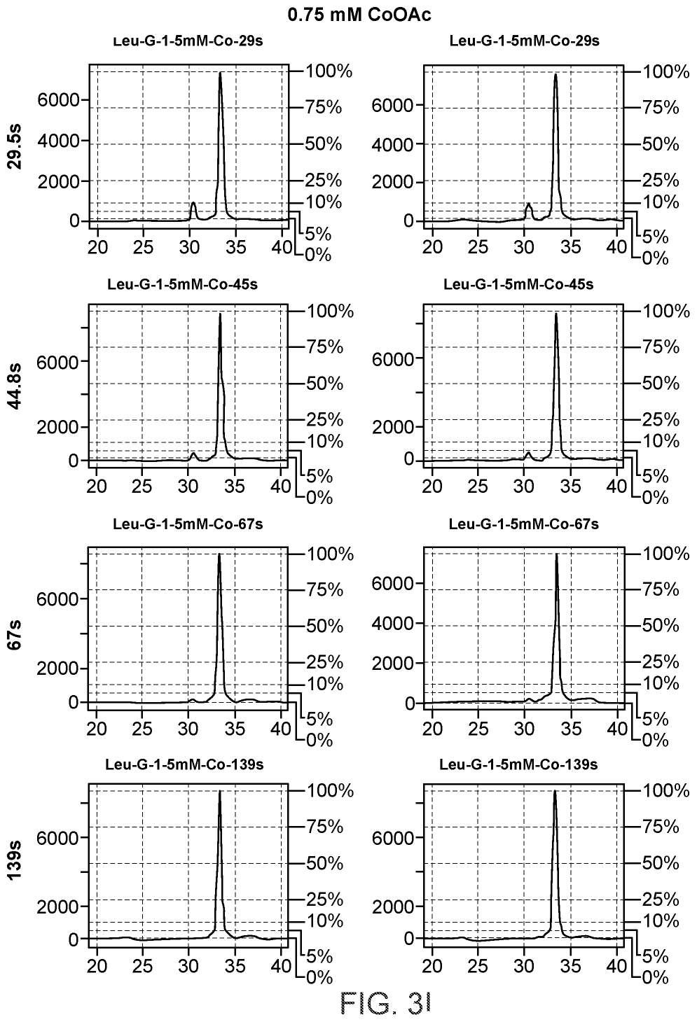

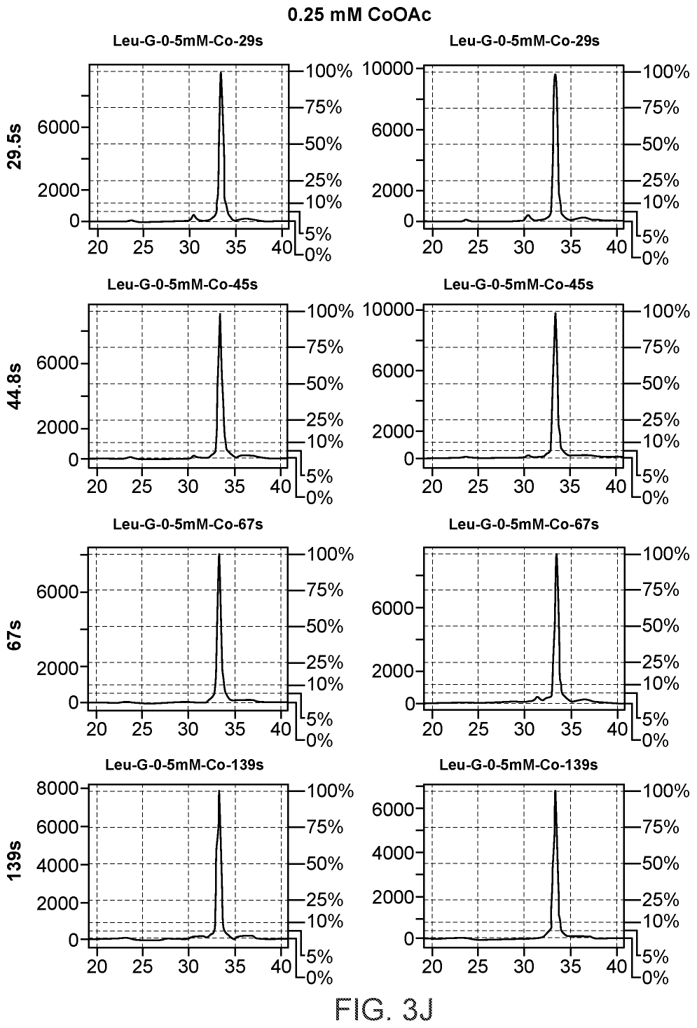

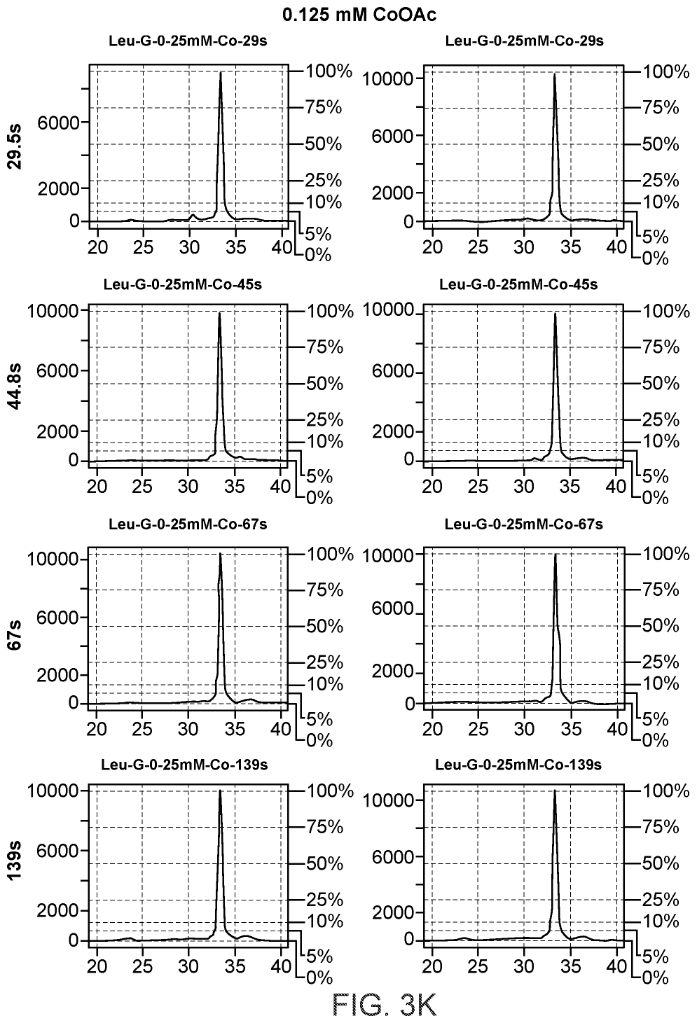

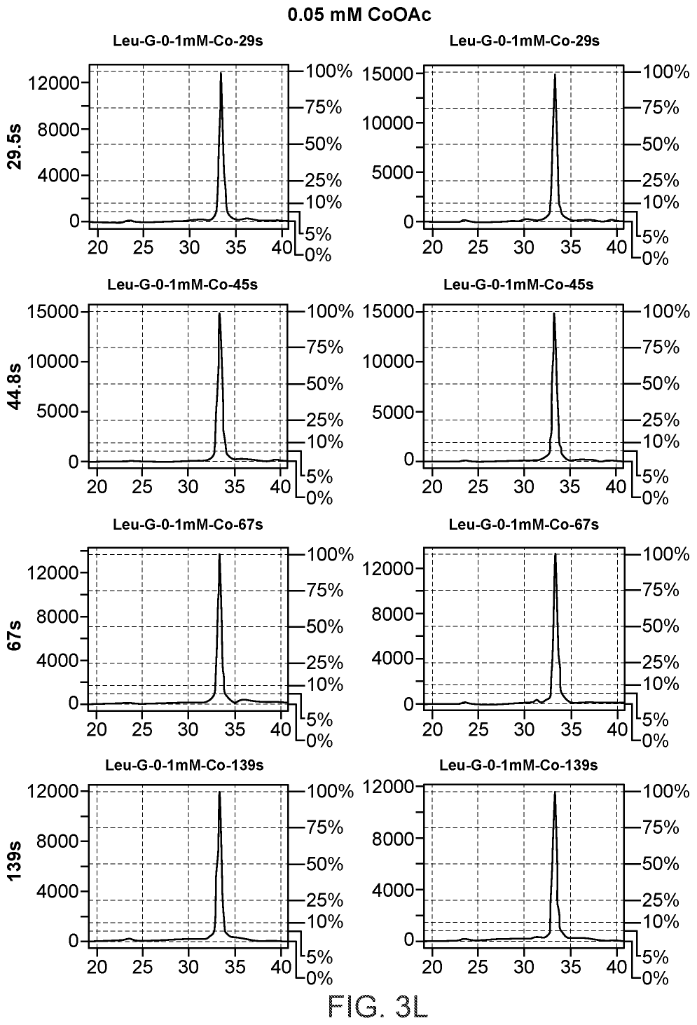

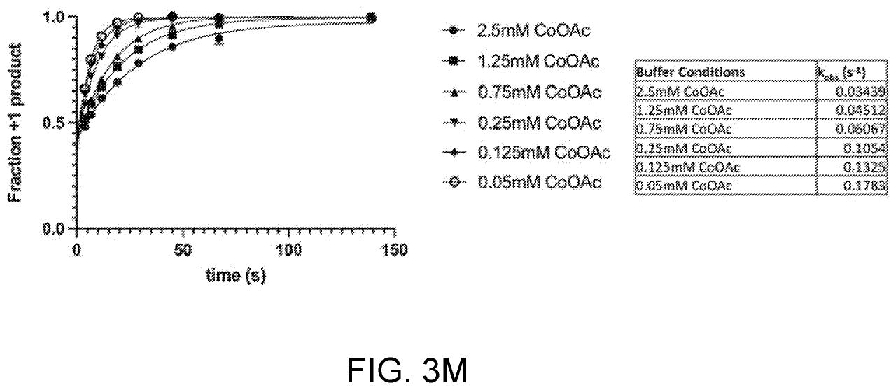

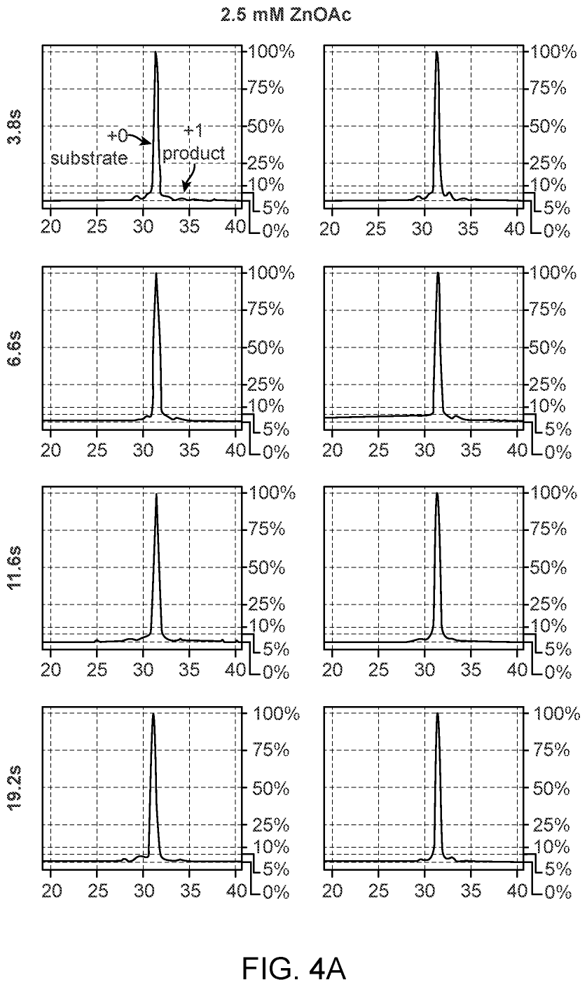

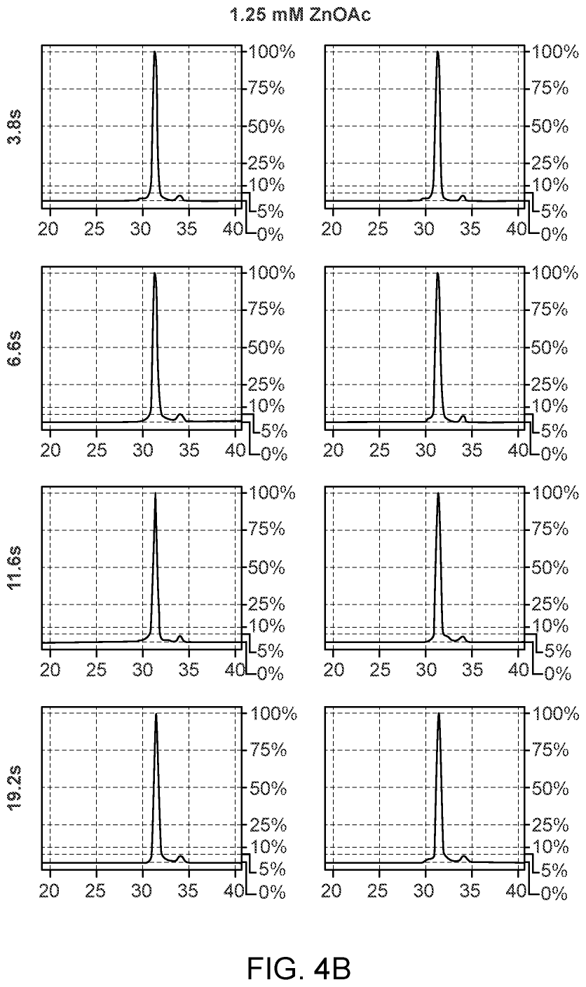

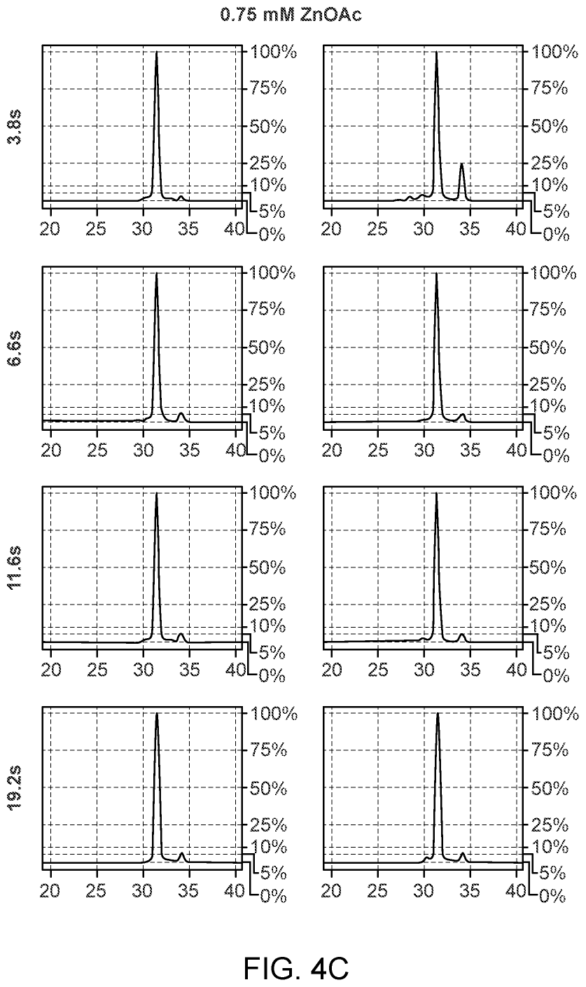

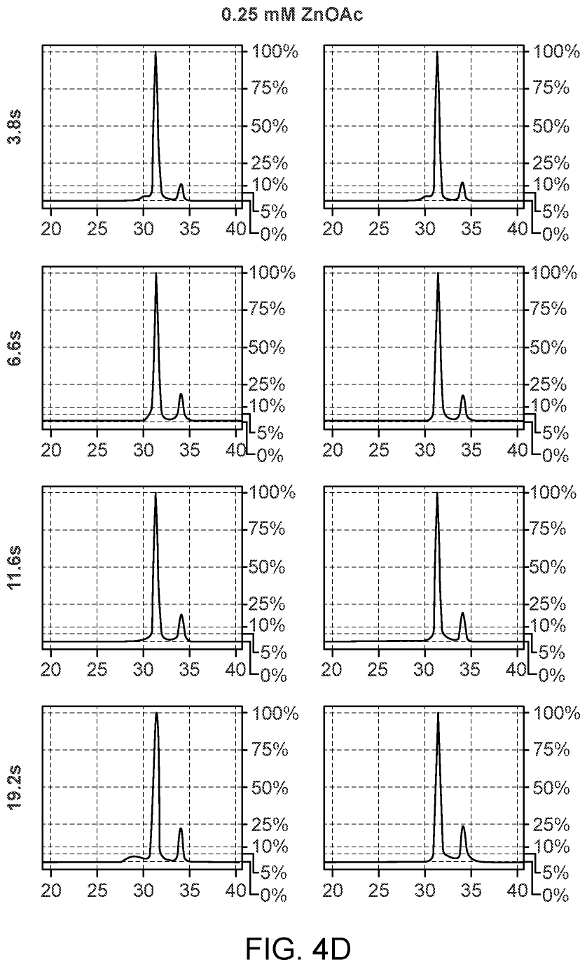

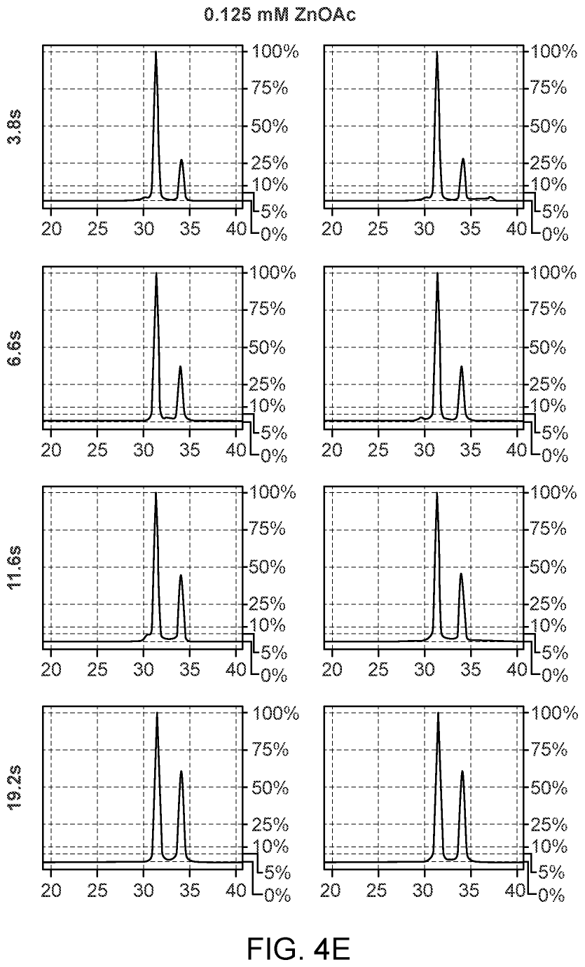

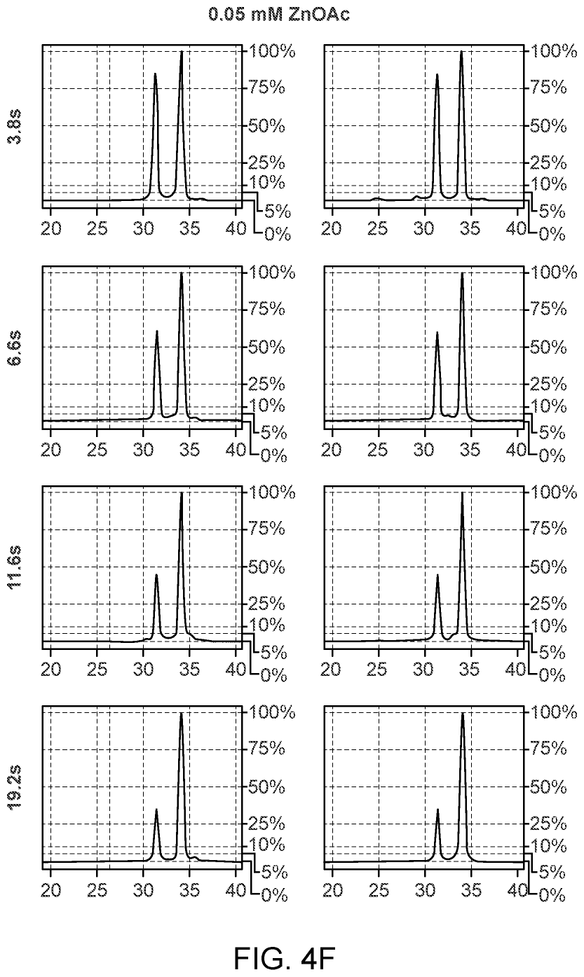

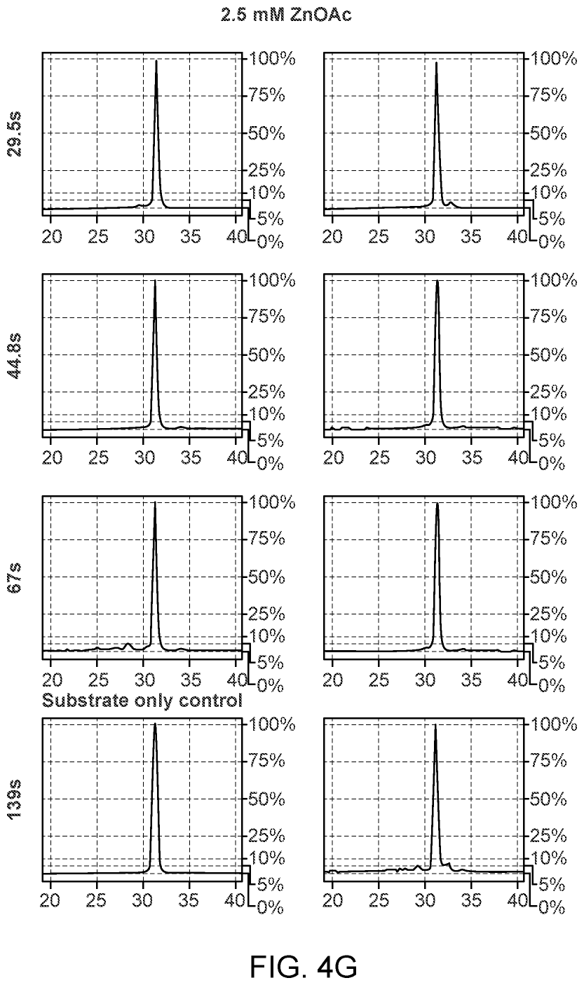

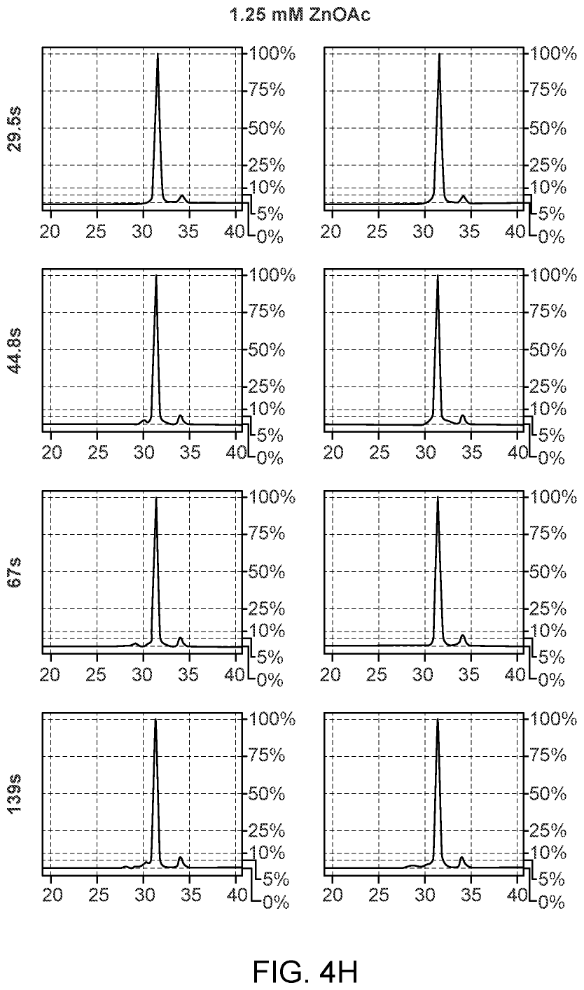

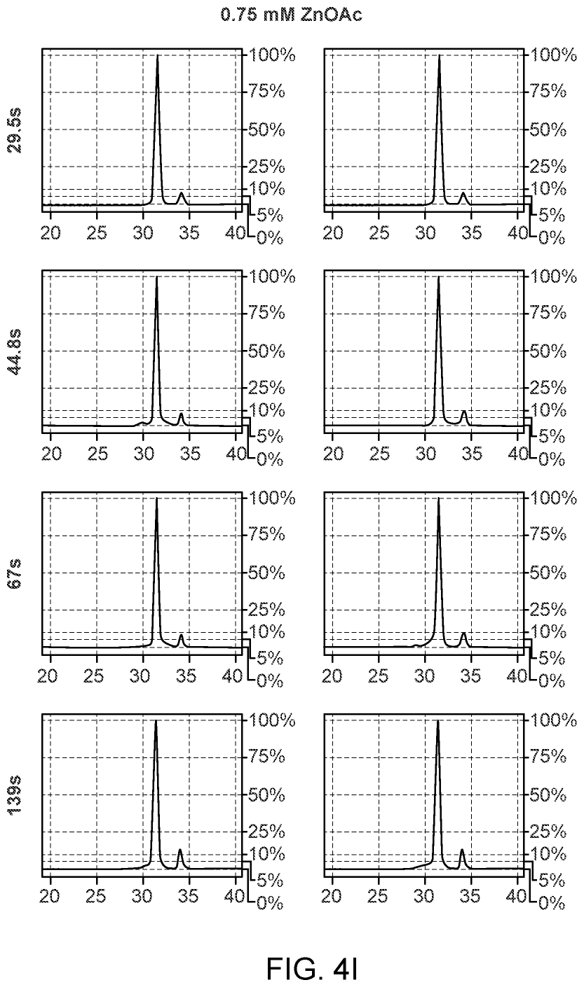

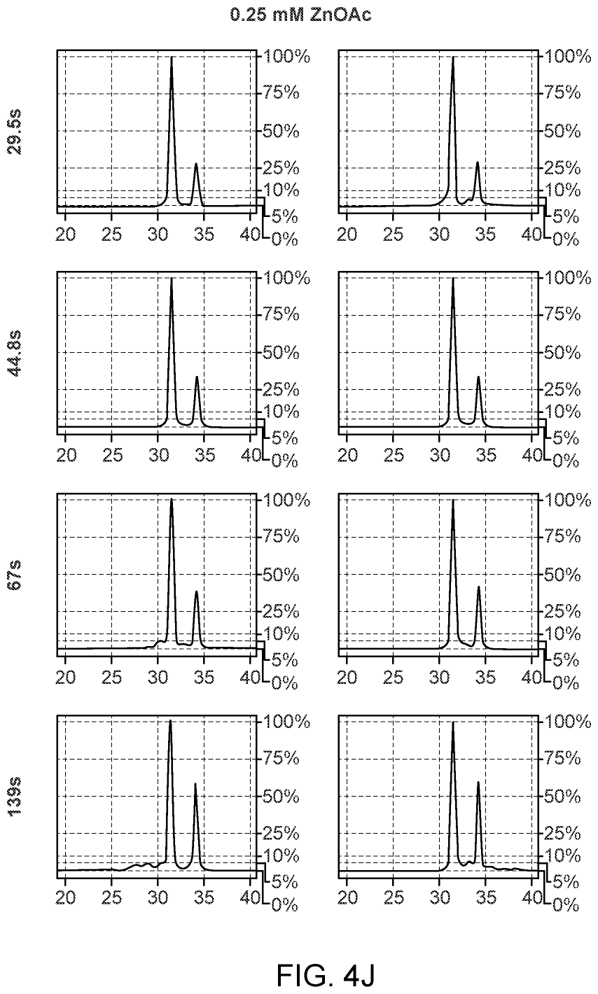

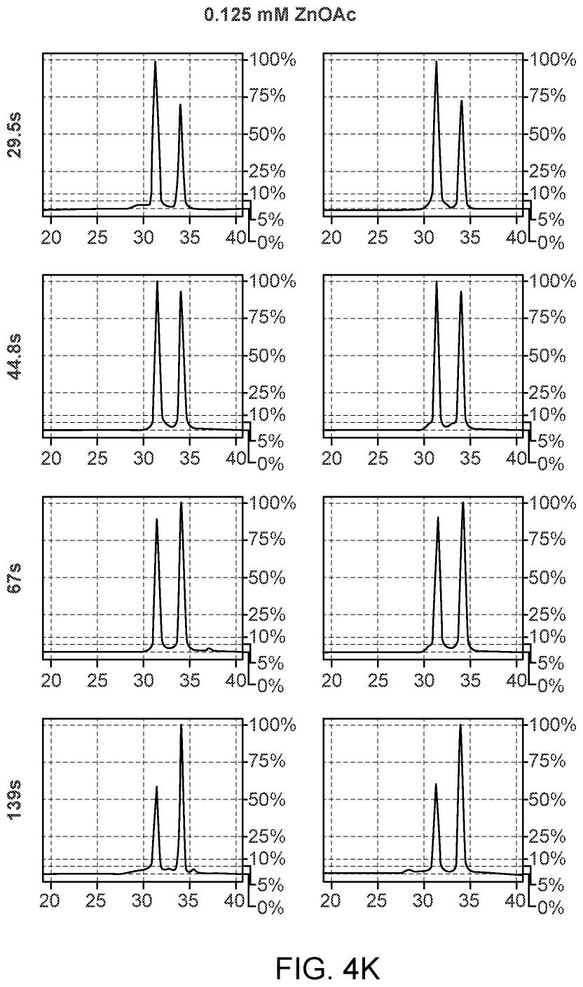

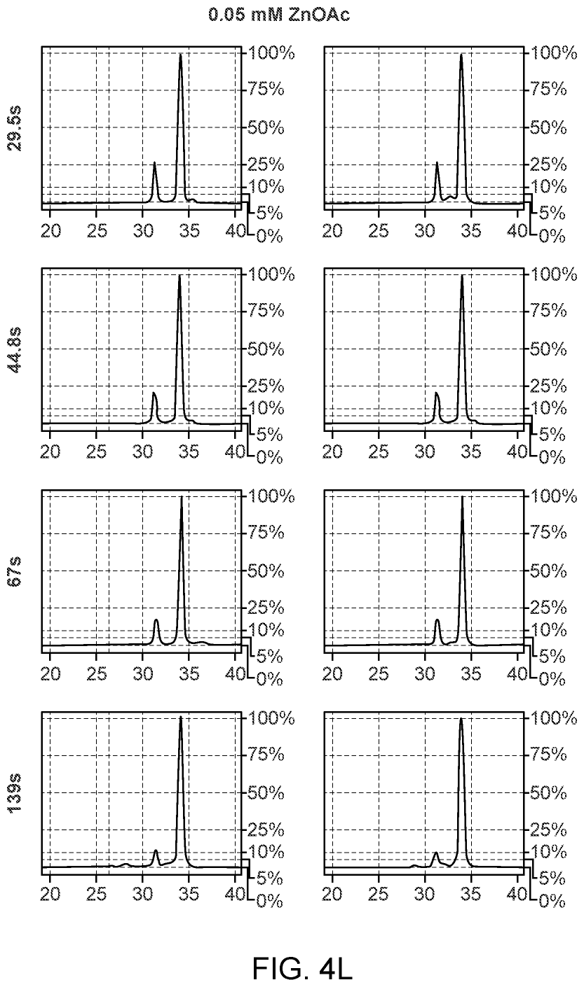

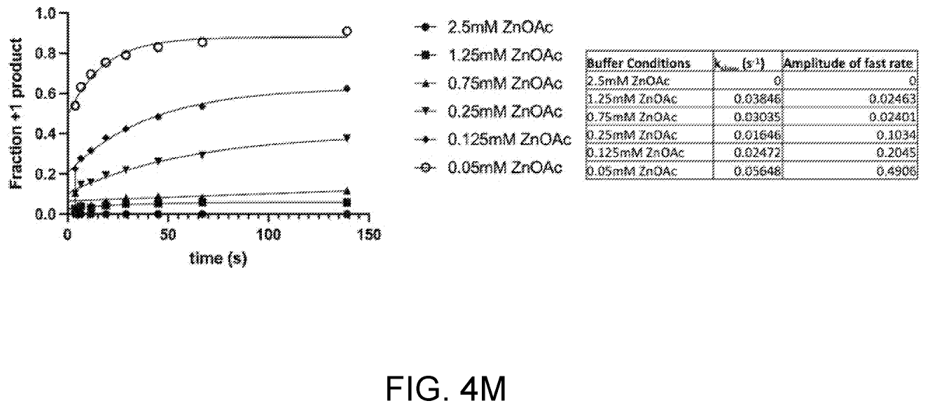

Analysis by capillary electrophoresis shows the abundance of the DNA oligo substrate (substrate) and the product containing the incorporated nucleotide (product) at various time points in a polynucleotide extension reaction including TdT enzyme conjugated to a G-nucleotide, in reaction buffer containing cobalt acetate or zinc acetate at different concentrations, from 50 μM-2.5 mM cobalt acetate (0.05 mM CoOAc, 0.125 mM CoOAc, 0.25 mM CoOAc, 0.75 mM CoOAc, 1.25 mM CoOAc, and 2.5 mM CoOAc; see FIGS. 3A-3L and Table 1) or 50 μM-2.5 mM zinc acetate (0.05 mM ZnOAc, 0.125 mM ZnOAc, 0.25 mM ZnOAc, 0.75 mM ZnOAc, 1.25 mM ZnOAc, and 2.5 mM ZnOAc; see FIGS. 4A-4L). The results indicate that nucleotide incorporation rates using TdT-nucleotide conjugates increase as a function of decreasing divalent metal ions concentrations. This was observed for both cobalt acetate and zinc acetate, as demonstrated by increased abundance of product observed at faster time points in lower concentrations of the divalent metal ion. In extension reactions containing standard cobalt acetate concentrations (2.5 mM), the DNA oligo substrate is not turned over completely after 139 seconds. In conditions using lower cobalt acetate conditions, the extension reaction is complete in less than 139 seconds (see Table 1).

Results from synthesis reactions shown in FIGS. 3A-3L were quantified and reaction rates were determined for each of the buffer conditions (FIG. 3M). The calculated reaction rates (k_{obs}) increased as the concentration of CoOAc decreased.

The results in FIGS. 4A-4L were quantified and reaction rates were determined for each of the buffer conditions (FIG. 4M). Turnover under these conditions exhibit biphasic behavior with an initial fast rate and a slow rate (k_{slow}). Amplitude of the initial fast rate is shown in the table in FIG. 4M. The rates of both the initial fast rate and the slow rate increased as the concentration of ZnOAc decreased.

TABLE 1
Nucleotide Incorporation Rate at Various CoOAc Concentrations

| Cobalt Acetate | Conjugated Nucleotide |
|---|---|
| Concentration (mM) | Incorporation Completion Time (s) |
| 0.05 | 29.5 |
| 0.125 | 44.8 |
| 0.25 | 67 |
| 0.75 | 139 |
| 1.25 | >139 |
| 2.5 | >139 |

Rate of Free Nucleotide Incorporation is Reduced with Decreasing Divalent Metal Concentration

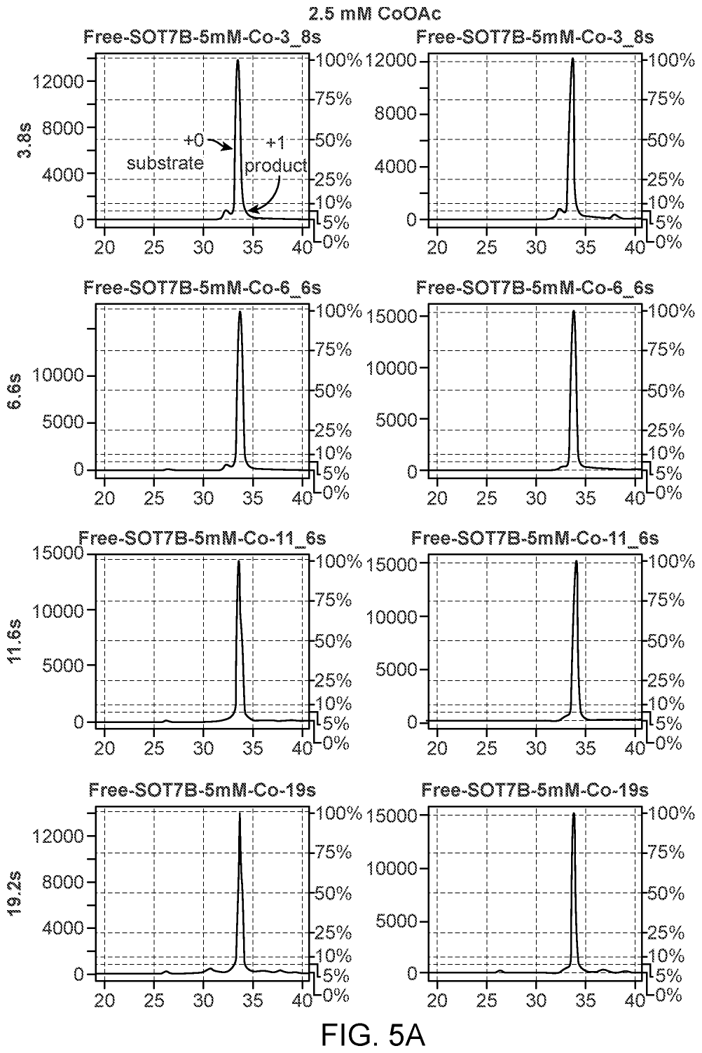

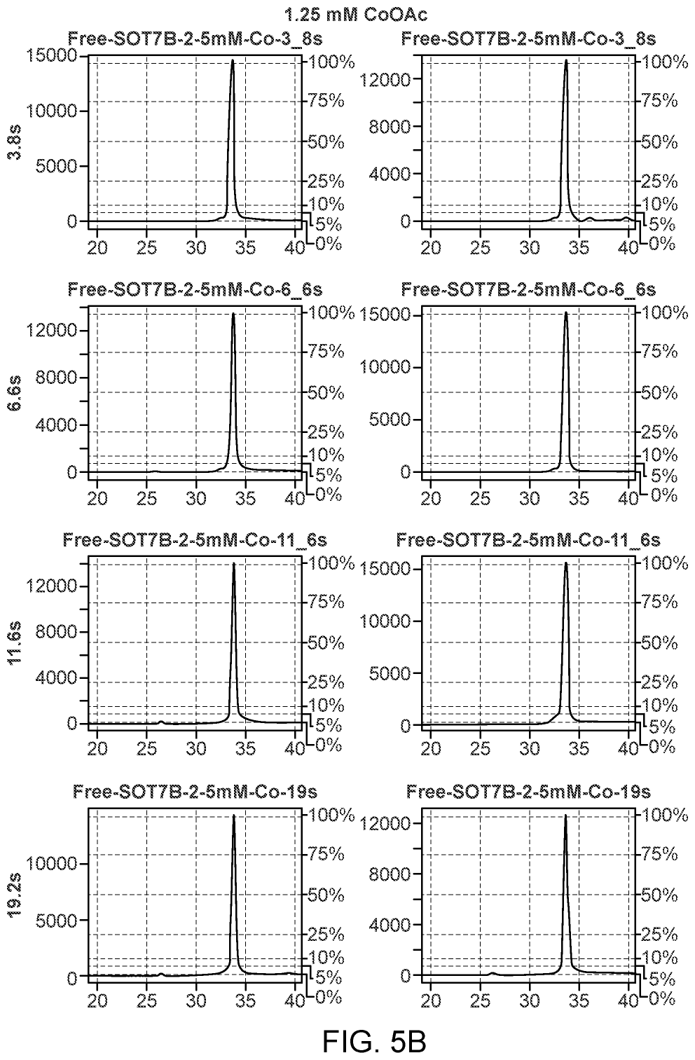

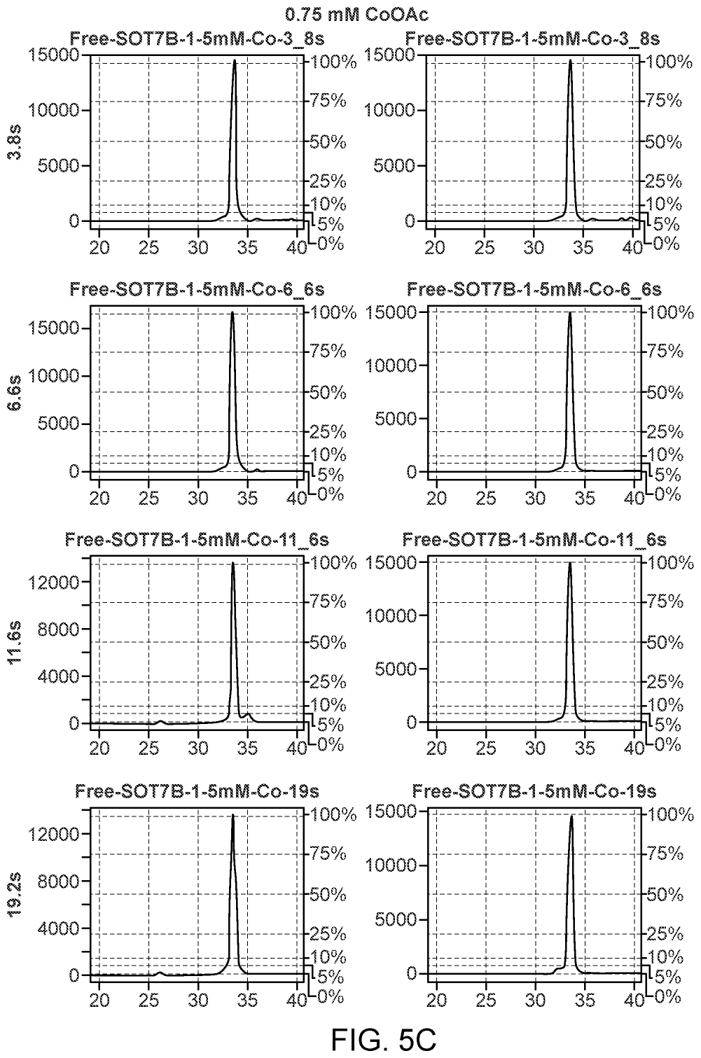

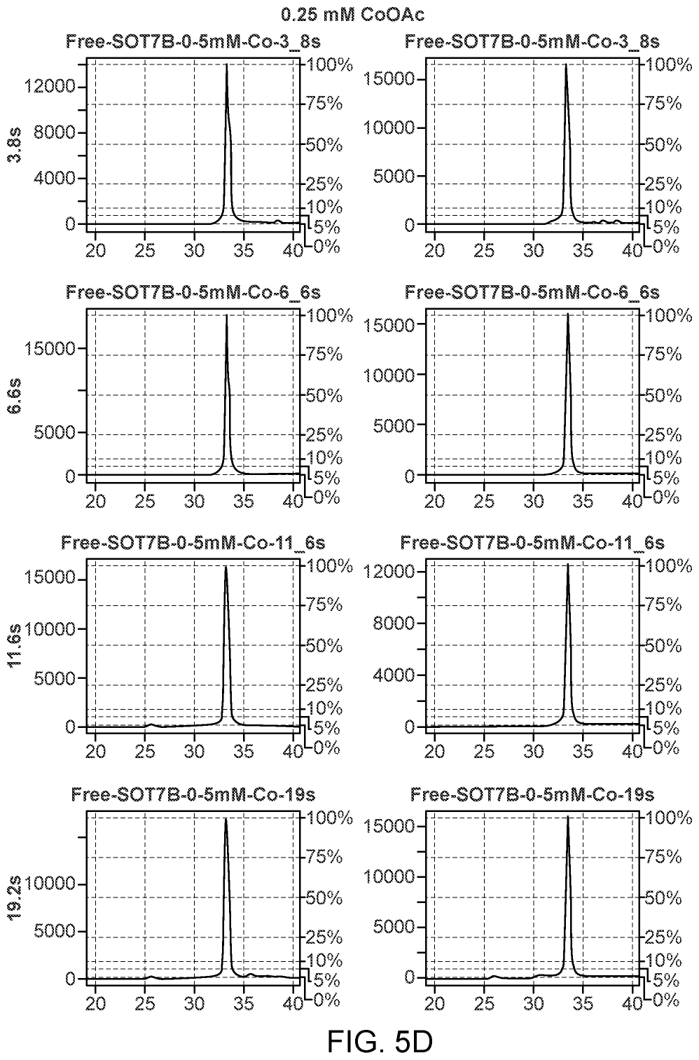

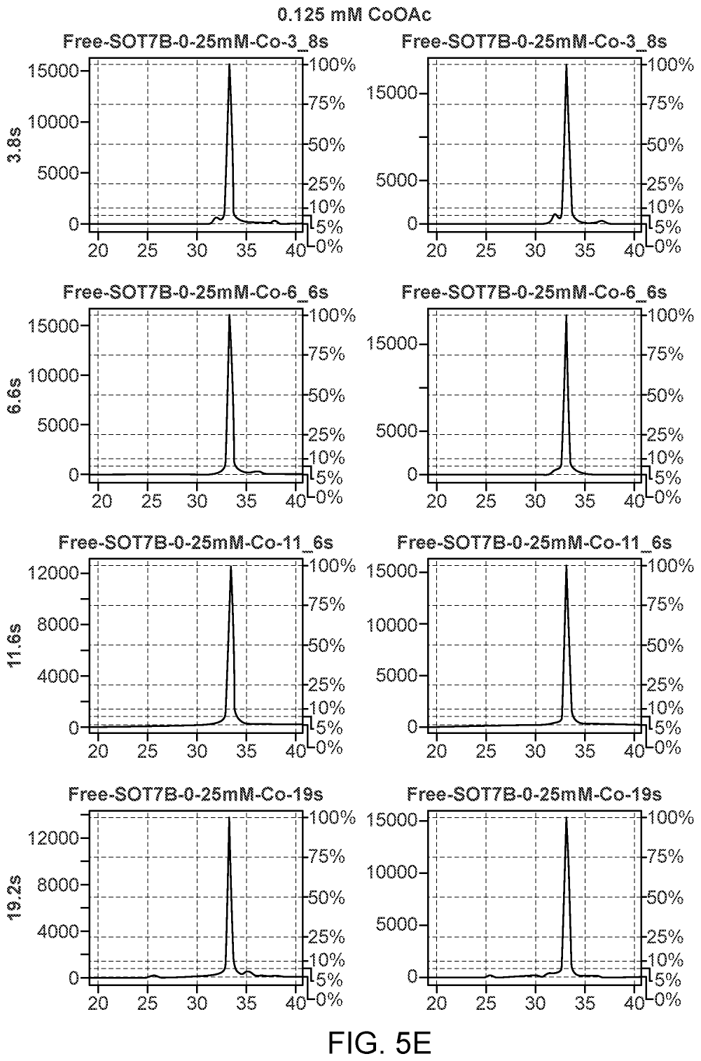

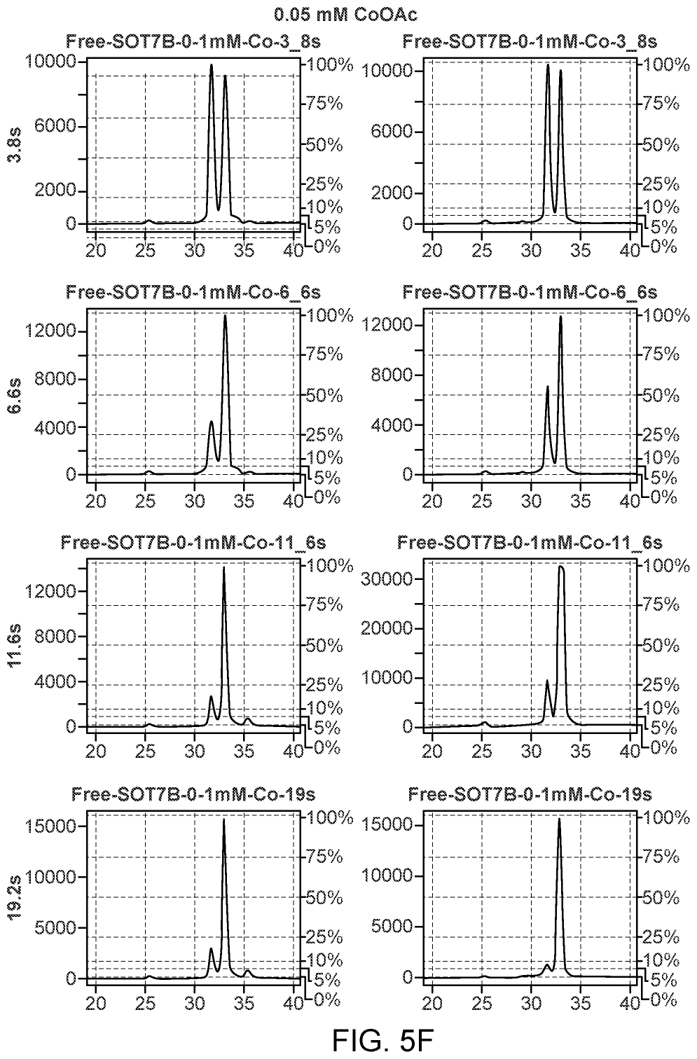

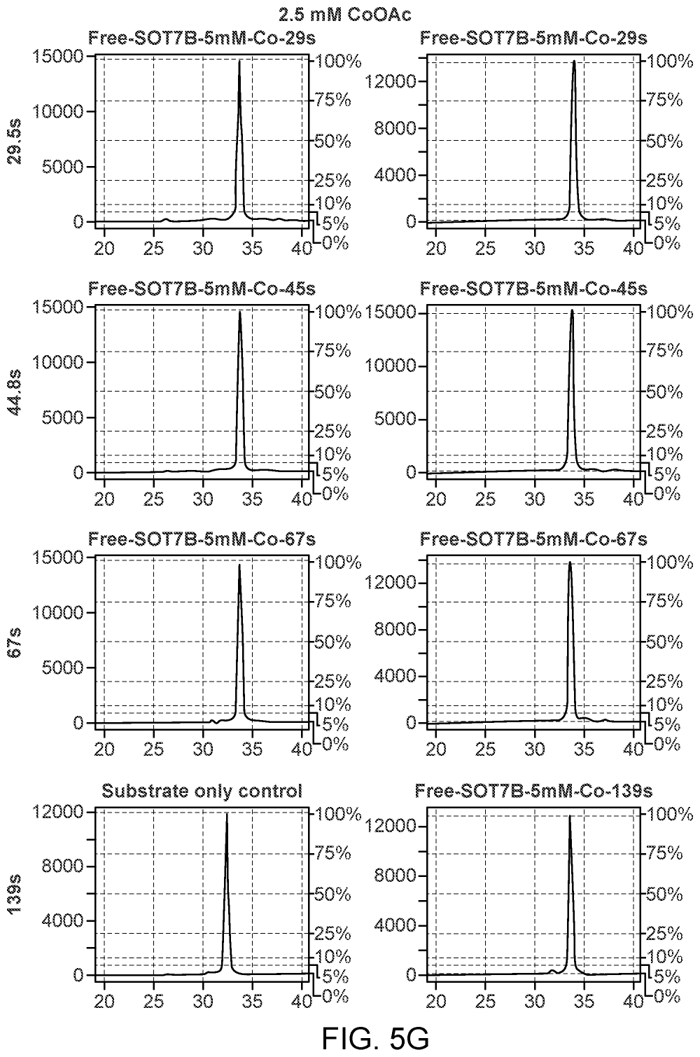

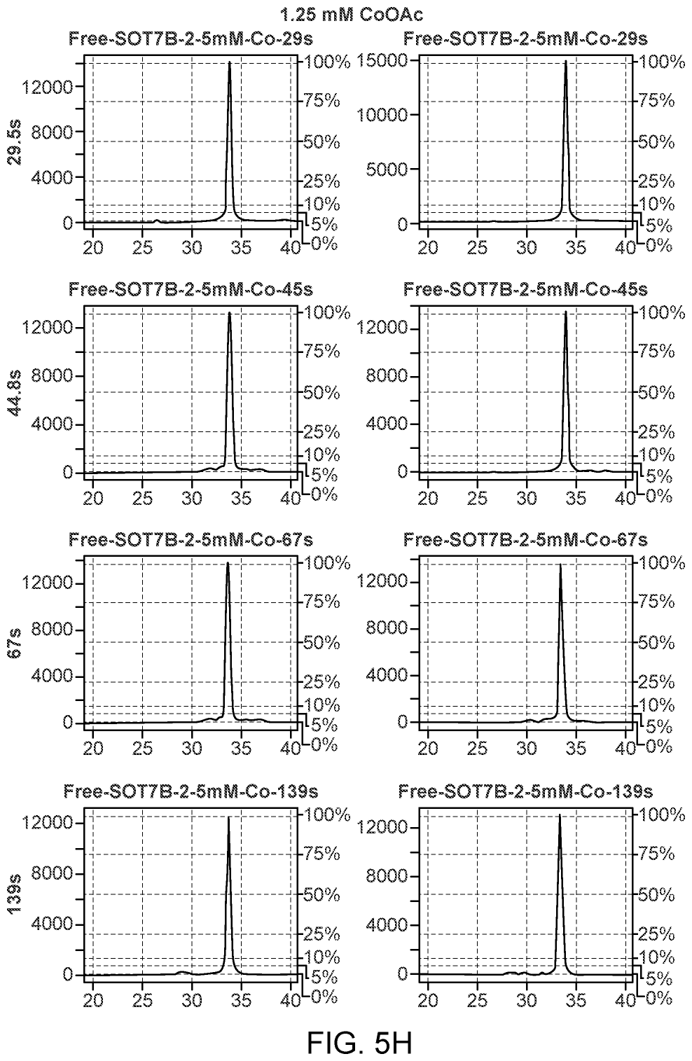

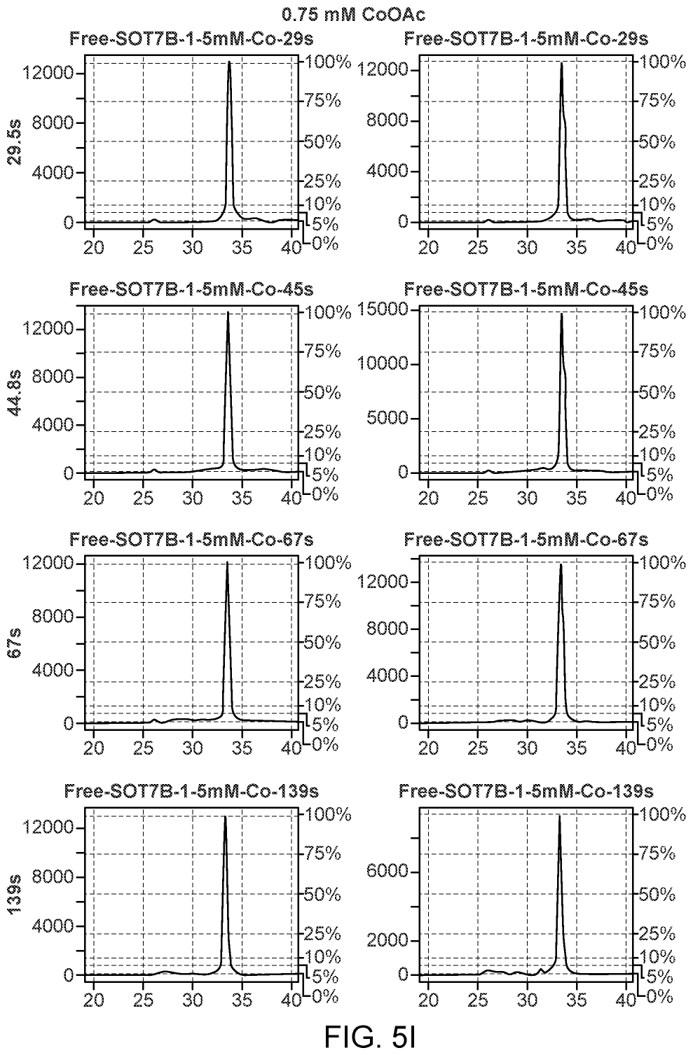

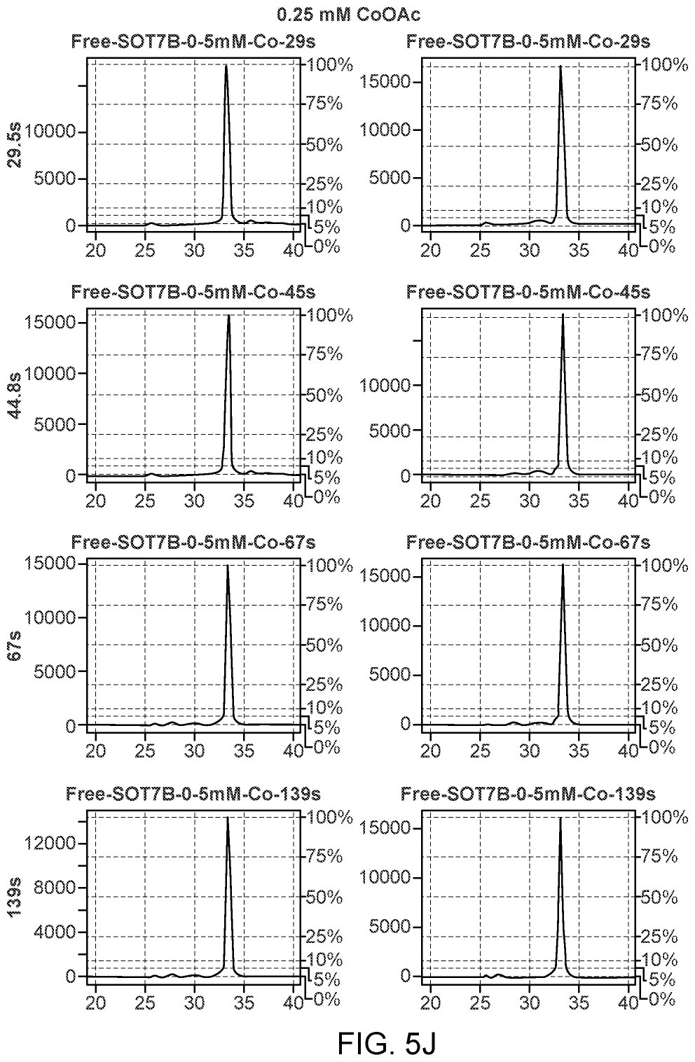

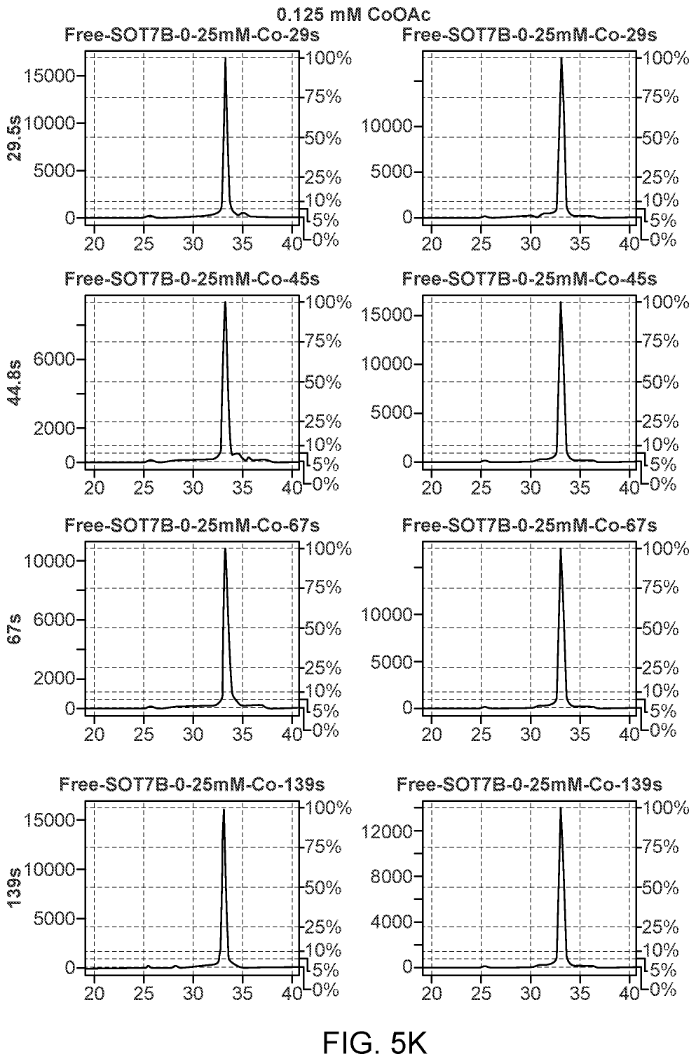

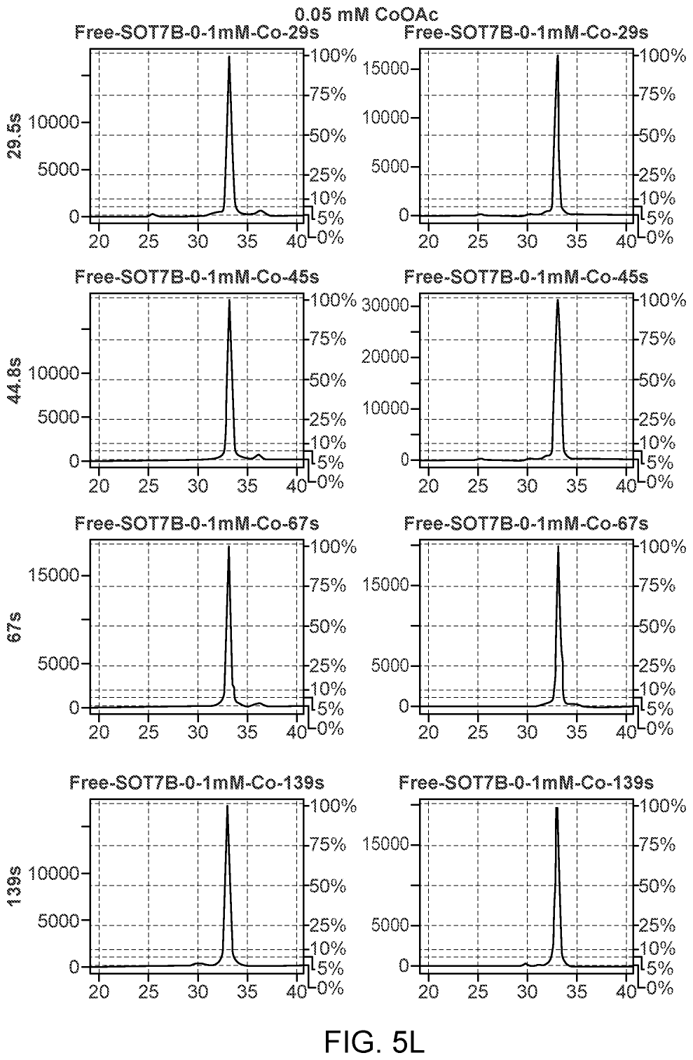

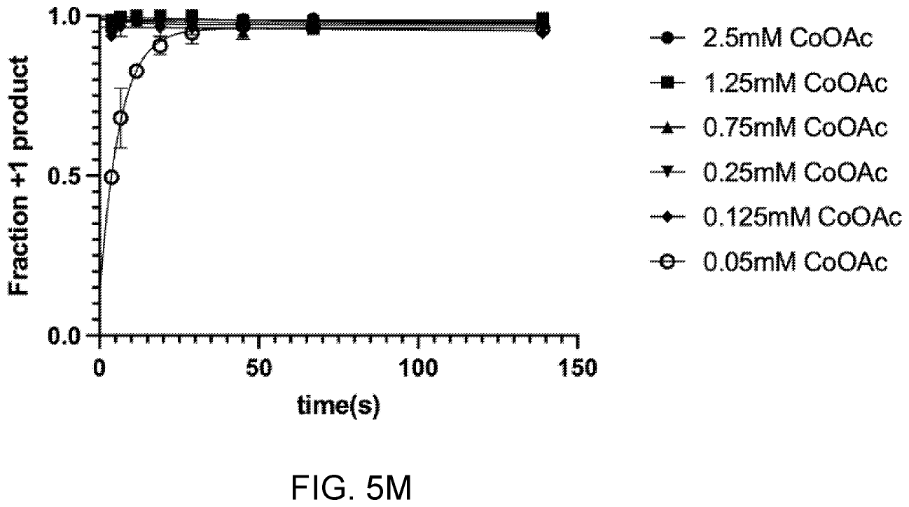

In this example, the nucleotide incorporation rates were analyzed for TdT enzyme catalyzed incorporation of a free G-nucleotide into an DNA oligo using a range of cobalt acetate concentrations in the absence of magnesium acetate (FIGS. 5A-5L).

Enzymatic polynucleotide synthesis was carried out in buffer containing 20 mM Tris Acetate, 50 mM Potassium Acetate, 50 μM-2.5 mM cobalt acetate (0.05 mM CoOAc, 0.125 mM CoOAc, 0.25 mM CoOAc, 0.75 mM CoOAc, 1.25 mM CoOAc, and 2.5 mM CoOAc; see FIGS. 5A-5L), 50 nM DNA oligo substrate, 1 μM TdT enzyme-nucleotide conjugate, free nucleotide substrate (ddGTP, 0.5 mM), pH 7.9. Polymerase-nucleotide conjugate kinetic activity was measured upon quenching reactions with EDTA and performing DNA fragment analysis by capillary electrophoresis.

FIGS. 5A-5L show abundance of the DNA oligo substrate (substrate) and the product containing the incorporated nucleotide (product) at various time points in a polynucleotide extension reaction including TdT enzyme and free ddGTP in reaction buffer containing 50 μM-2.5 mM cobalt acetate (0.05 mM CoOAc, 0.125 mM CoOAc, 0.25 mM CoOAc, 0.75 mM CoOAc, 1.25 mM CoOAc, and 2.5 mM CoOAc). The results in FIGS. 5A-5L were quantified and plotted, the results show a slower reaction rate for the lowest concentration of CoOAc (FIG. 5M).

Unlike nucleotide incorporation mediated by a TdT-nucleotide conjugate where the effective nucleotide concentration relative to the polymerase is artificially increased, reducing cobalt acetate concentrations in the presence of free TdT and free nucleotide dramatically reduced the nucleotide incorporation rate (Table 2).

TABLE 2
Nucleotide Incorporation Rate at Various CoOAc Concentrations

| | Free Nucleotide Incorporation |
|---|---|
| Cobalt Acetate (mM) | Completion Time (s) |
| 0.05 | 29.5 |
| 0.125 | 6.6 |
| 0.25 | 6.6 |
| 0.75 | 6.6 |
| 1.25 | 6.6 |
| 2.5 | 6.6 |

### Summary of Results

Taken together, the results show that, surprisingly, as compared to standard divalent metal ion concentrations, low divalent metal ion concentrations improve nucleotide incorporation rate in the extension of a polynucleotide by a TdT-nucleotide conjugate including by increasing rate of nucleotide incorporation in a polynucleotide. That is, in contrast to previously documented results showing that incorporation rate of nucleotides increase at standard concentrations of divalent metal ions (about 2.5 mM for most divalent metal ions), the present results provide unexpected findings demonstrating that low divalent metal ion concentrations increase nucleotide incorporation rates in extension reactions. The standard concentrations are supported by findings in the literature (Kato et al. J Biol. Chem. 242(11). 1967) and suggested by protocols included with commercially available polymerases and corresponding reaction buffers (see, e.g., protocols and buffer from ThermoFisher https://www.thermofisher.com/order/catalog/product/16314015). In contrast, the results here show that divalent metal ions lower than the standard concentrations increase nucleotide incorporation using polymerase-nucleotide conjugates.

Examples shown here were performed with dGTP. Similarly, for other nucleobases the low concentration of divalent metal ion improved extension reactions (data not shown). Further, the effect of low cobalt and zinc concentrations with TdT-nucleotide conjugates may be generalized to other polymerase-nucleotide conjugates, as well as other appropriate divalent cations (e.g. Mg²⁺, Ca²⁺, Sr²⁺, Ba²⁺, Mn²⁺, Co²⁺, Fe²⁺, Ni²⁺, Cu²⁺, Zn²⁺) used in a nucleotide incorporation reaction.

### Other Embodiments

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method of nucleic acid synthesis, comprising:
providing a conjugate reagent comprising a plurality of polymerase-nucleotide conjugates,
wherein said conjugates comprise a nucleotide attached to a polymerase via a linker; and
providing a sample comprising a polynucleotide;
contacting said sample with said conjugate reagent in a reaction volume, such that said sample and said conjugate reagent are combined and in the presence of one another,
wherein said polymerase of the conjugate catalyzes an extension reaction comprising covalent addition of said nucleotide of said conjugate onto the 3′ hydroxyl of said polynucleotide,
and wherein at least one divalent cation is present in said reaction volume; and
wherein a total concentration of divalent cations present in the reaction volume is no greater than about 500 μM.

2. The method of claim 1, wherein the total concentration of divalent cations present in the reaction volume is no greater than about 250 μM, about 125 μM or about 50 μM.

3. The method of claim 1 or claim 2, wherein the extension reaction performed has a faster turnover rate than an extension reaction performed in the presence of the same or another divalent cation at a concentration of greater than about 1000 μM.

4. The method of claim 3, wherein the faster turnover rate is about is about 1 second faster, about 2 seconds faster, about 3 seconds faster, about 4 seconds faster, about 5 seconds faster, about 6 seconds faster, about 7 seconds faster, about 8 seconds faster, about 9 seconds faster, about 10 seconds faster, about 15 seconds faster, about 20 seconds faster, about 25 seconds faster, about 30 seconds faster, about 35 seconds faster, about 40 seconds faster, about 45 seconds faster, about 50 seconds faster, about 55 seconds faster, or about 60 seconds faster as compared to a reference reaction performed in the presence of the divalent cation at a concentration greater than about 1000 μM.

5. The method of claim 1, wherein the divalent cation present at the highest concentration in the reaction volume is cobalt (Co²⁺) or zinc (Zn²⁺).

6. A method of nucleic acid synthesis, comprising:
providing a conjugate reagent comprising a plurality of polymerase-nucleotide conjugates,
wherein said conjugates comprise a nucleotide attached to a polymerase via a linker, and
contacting a sample comprising a polynucleotide with said conjugate reagent,
wherein said polymerase of said conjugate catalyzes an extension reaction comprising covalent addition of said nucleotide of said conjugate onto the 3′ hydroxyl of said polynucleotide in the presence of at least one divalent cation, and
wherein the at least one divalent cation concentration is less than about 2500 μM.

7. The method of claim 5, wherein the at least one divalent cation is present at a concentration no greater that about 250 μM, and wherein the extension reaction performed in the presence of the concentration no greater than about 2500 μM has a faster turnover rate than an extension reaction performed in the presence of the same or another divalent cation at a concentration of greater than about 2500 μM.

8. The method of claim 6 or claim 7, wherein the concentration no greater than about 2500 μM is about 1 μM, about 2.5 μM, about 5 μM, about 10 μM, about 15 μM, about 20 μM, about 25 μM, about 30 μM, about 35 μM, about 40 μM, about 4 μM, about 50 μM, about 100 μM, about 150 μM, about 200 μM, about 250 μM, about 300 μM, about 350 μM, about 400 μM, about 450 μM, about 500 μM, about 750 μM, about 1000 μM, about 1500 μM, about 2000 μM, or up to about 2500 μM.

9. The method of claim 6 or 7, wherein the faster turnover rate is between about 60 seconds faster and about 300 seconds faster as compared to a reference reaction performed in the presence of a divalent cation concentration greater than about 2500 μM.

10. The method of any one of claims 6-9, wherein the faster turnover rate is about is about 1 second faster, about 2 seconds faster, about 3 seconds faster, about 4 seconds faster, about 5 seconds faster, about 6 seconds faster, about 7 seconds faster, about 8 seconds faster, about 9 seconds faster, about 10 seconds faster, about 15 seconds faster, about 20 seconds faster, about 25 seconds faster, about 30 seconds faster, about 35 seconds faster, about 40 seconds faster, about 45 seconds faster, about 50 seconds faster, about 55 seconds faster, or about 60 seconds faster as compared to a reference reaction performed in the presence of the divalent cation at a concentration of greater than about 2500 μM.

11. The method of any one of claims 6-10, wherein the at least one divalent cation does not comprise Mg²⁺, Ca²⁺, Sr²⁺, Ba²⁺, Mn²⁺, Co²⁺, Fe²⁺, Ni²⁺, Cu²⁺, and Zn²⁺.

12. The method of any one of claims 6-11, wherein the at least one divalent cation is Co²⁺.

13. The method of any one of claims 6-11, wherein the at least one divalent cation is Zn²⁺.

14. The method of any one of claims 6-11, wherein the extension reaction is carried out in the absence of Mg²⁺.

15. The method of any one of claims 1-14, wherein the nucleotide is a modified nucleotide.

16. The method of any one of claims 1-15, wherein the linker is a cleavable linker.

17. The method of 16, further comprising cleaving the linker.

18. A method of synthesizing a polynucleotide comprising repeating the method of claim 1 or claim 6, followed by the method of claim 16 one or more times.

19. The method of claim 18, wherein the polynucleotide comprises a pre-determined sequence.

20. The method of any one of the preceding claims, wherein the polymerase is a template-independent polymerase.

21. The method of claim 20, wherein the polymerase is selected from a Pol IV, a Pol μ, and a terminal deoxyribonucleotidyl transferase (TdT), or a variant thereof.

22. The method of claim 21, wherein the polymerase is a TdT, or a variant thereof.

23. The method of any one of claims 1-19, wherein the polymerase is a template-dependent polymerase.

24. The method of claim 23, wherein the polymerase is a DNA polymerase.

25. The method of claim 23, wherein the polymerase is an RNA polymerase.

26. A nucleic acid synthesis reaction buffer comprising at least one divalent metal ion, wherein when used in a synthesis reaction, the concentration of the divalent metal ion present when the synthesis reaction is being performed is no greater than about 2500 μM.

27. The nucleic acid synthesis reaction buffer of claim 26, wherein the at least one divalent cation present in the synthesis reaction is less than about 1 μM, about 2.5 μM, about 5 μM, about 10 μM, about 15 μM, about 20 μM, about 25 μM, about 30 μM, about 35 μM, about 40 μM, about 45 μM, about 50 μM, about 100 μM, about 150 μM, about 200 μM, about 250 μM, about 300 μM, about 350 μM, about 400 μM, about 450 μM, about 500 μM, about 750 μM, about 1000 μM, about 1500 μM, about 2000 μM, or up to about 2500 μM.

28. The nucleic acid synthesis reaction buffer of claim 26 or claim 27, wherein the at least one divalent cation is selected from Mg²⁺, Ca²⁺, Sr²⁺, Ba²⁺, Mn²⁺, Co²⁺, Fe²⁺, Ni²⁺, Cu²⁺, and Zn²⁺, or a combination thereof.

29. The nucleic acid synthesis reaction buffer of claim 26 or 27, wherein the at least one divalent cation is Mg²⁺.

30. The nucleic acid synthesis reaction buffer of claim 26 or 27, wherein the at least one divalent cation is Ca²⁺.

31. The nucleic acid synthesis reaction buffer of claim 26 or 27, wherein the at least one divalent cation is Sr²⁺.

32. The nucleic acid synthesis reaction buffer of claim 26 or 27, wherein the at least one divalent cation is Ba²⁺.

33. The nucleic acid synthesis reaction buffer of claim 26 or 27, wherein the at least one divalent cation is Mn²⁺.

34. The nucleic acid synthesis reaction buffer of claim 26 or 27, wherein the at least one divalent cation is Co²⁺.

35. The nucleic acid synthesis reaction buffer of claim 26 or 27, wherein the at least one divalent cation is Fe²⁺.

36. The nucleic acid synthesis reaction buffer of claim 26 or 27, wherein the at least one divalent cation is Ni²⁺.

37. The nucleic acid synthesis reaction buffer of claim 26 or 27, wherein the at least one divalent cation is Cu²⁺.

38. The nucleic acid synthesis reaction buffer of claim 26 or 27, wherein the at least one divalent cation is Zn²⁺.

39. The nucleic acid synthesis reaction buffer of any one of claims 26-38, further comprising tris(hydroxymethyl)aminomethane.

40. The nucleic acid synthesis reaction buffer of any one claims 26-39, further comprising 4-(2-hydroxyethyl)-1-piperazineethanesulfonic acid.

41. The nucleic acid synthesis reaction buffer of any one claims 26-40, further comprising a potassium salt.

42. The nucleic acid synthesis reaction buffer of any one of claims 26-41, further comprising a nonionic surfactant.

43. The nucleic acid synthesis reaction buffer of any one of claims 26-42, further comprising bovine serum albumin.

44. The nucleic acid synthesis reaction buffer of any one of any one of claims 26-43, further comprising sodium chloride.

45. The nucleic acid synthesis reaction buffer of any one of any one of claims 26-44, further comprising β-mercaptoethanol.

46. The nucleic acid synthesis reaction buffer of any one of claims 26-45, further comprising glycerol.

47. A method of nucleic acid synthesis, comprising:
providing a conjugate reagent comprising a plurality of polymerase-nucleotide conjugates,
wherein said conjugates comprise a nucleotide covalently linked to a polymerase via a linker, and
contacting a sample comprising a polynucleotide with said conjugate reagent,
wherein said polymerase of the conjugate catalyzes an extension reaction comprising the covalent addition of the nucleotide of the conjugate onto the 3′ hydroxyl of said polynucleotide in the nucleic acid synthesis reaction buffer of any one of claims 26-46.

48. The method of 47, wherein the nucleotide is a modified nucleotide.

49. The method of claim 47 or claim 48, wherein the linker is a cleavable linker.

50. The method of claim 49, further comprising cleaving the linker.

51. The method of claim 50, further comprising repeating each of the steps to synthesize a polynucleotide.

52. The method of claim 51, wherein the polynucleotide comprises a pre-determined sequence.

53. The method of any one of claims 47-52, wherein the polymerase is a template-independent polymerase.

54. The method of claim 53, wherein the polymerase is selected from a Pol IV, a Pol μ, and a terminal deoxyribonucleotidyl transferase (TdT), or a variant thereof.

55. The method of claim 54, wherein the polymerase is a TdT, or a variant thereof.

56. The method of any one of claims 47-42, wherein the polymerase is a template-dependent polymerase.

57. The method of claim 56, wherein the polymerase is a DNA polymerase.

58. The method of claim 56, wherein the polymerase is an RNA polymerase.

59. A method of performing a nucleic acid synthesis reaction comprising providing a conjugate reagent comprising a plurality of polymerase-nucleotide conjugates, wherein said conjugates comprise a nucleotide covalently linked to a polymerase via a linker, and contacting a sample comprising a polynucleotide with said conjugate reagent, wherein said polymerase of the conjugate catalyzes an extension reaction comprising the covalent addition of the nucleotide of the conjugate onto the 3′ hydroxyl of said polynucleotide in the presence of a concentration of cobalt (Co²⁺), and, optionally, in the absence of Mg²⁺, wherein the concentration of Co²⁺ is selected from about 0.250 mM Co²⁺, about 0.125 mM Co²⁺, or about 0.050 mM Co²⁺, and wherein the extension reaction in the presence of the about 0.250 mM Co²⁺, about 0.125 mM Co²⁺, or about 0.050 mM Co²⁺ is faster than a reaction performed in the presence of a concentration of greater than 0.500 mM Co²⁺.

60. A method of performing a nucleic acid synthesis reaction comprising providing a conjugate reagent comprising a plurality of polymerase-nucleotide conjugates, wherein said conjugates comprise a nucleotide covalently linked to a polymerase via a linker, and contacting a sample comprising a polynucleotide with said conjugate reagent, wherein said polymerase of the conjugate catalyzes an extension reaction comprising the covalent addition of the nucleotide of the conjugate onto the 3′ hydroxyl of said polynucleotide in the presence of a concentration of zinc (Zn²⁺), and, optionally, in the absence of Mg²⁺, wherein the concentration of zinc is selected from about 0.250 mM Zn²⁺, about 0.125 mM Zn²⁺, or about 0.050 mM Zn²⁺, and wherein the extension reaction in the presence of the about 0.250 mM Zn²⁺, about 0.125 mM Zn²⁺, or about 0.050 mM Zn²⁺ is faster than a reaction performed in the presence of a concentration of greater than 0.500 mM Zn²⁺.

61. A method of reducing free nucleotide incorporation in a polynucleotide generated using a nucleic acid synthesis reaction comprising providing a conjugate reagent comprising a plurality of polymerase-nucleotide conjugates, wherein said conjugates comprise a nucleotide covalently linked to a polymerase via a linker, and contacting a sample comprising a polynucleotide with said conjugate reagent, wherein said polymerase of the conjugate catalyzes an extension reaction comprising the covalent addition of the nucleotide of the conjugate onto the 3′ hydroxyl of said polynucleotide in the presence of a concentration of cobalt (Co²⁺), and, optionally, in the absence of Mg²⁺, wherein the concentration of Co²⁺ is about 0.050 mM Co²⁺, and wherein the incorporation of free nucleotides into a polynucleotide using a nucleotide extension reaction in the presence of the about 0.050 mM Co²⁺ is reduced than in a reaction performed in the presence of a concentration of greater than 0.125 mM Co²⁺.

62. A method of improving a nucleotide synthesis reaction, the improvement comprising performing the nucleic acid synthesis in the presence of a divalent cation at a concentration of about 0.250 mM, about 0.125 mM, or about 0.050 mM of the divalent cation, wherein the speed of the extension reaction is greater than the speed of an extension reaction performed in the presence of a divalent cation at a concentration greater than about 0.500 mM.


SEQUENCE LISTING (2 sequences)

SEQ ID NO: 1 - protein, 391 aa, synthetic construct
MGGRDIVDGSEFSPSPVPGSQNVPAPAVKKISQYACQRRTTLNNYNQLFTDALDILAENDELRENEGSALAFMRASSVLKSLPFPITSMKDTEGIPSLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYADILESTFEKFKQPSRKVDALDHFQKCFLILKLDHGRVHSEKSGQQEGKGWKAIRVDLVMSPYDRRAFALLGWTGSRQFERDLRRYATHERKMMLDNHALYDRTKRVFLEAESEEEIFAHLGLDYIEPWERNA

SEQ ID NO: 2 - protein, 6 aa, synthetic construct
HHHHHH